(19) United States
(12) Patent Application Publication
(10) Pub. No.: US 2019/0225578 A1
(43) Pub. Date: Jul. 25, 2019

(54) PROCESS FOR TREATING KERATIN FIBRES WITH A CATIONIC DISULFIDE COMPOUND

(71) Applicant: L'OREAL, Paris (FR)

(72) Inventors: Christian BLAISE, Aulnay-sous-Bois (FR); Virginie BURCKBUCHLER, Aulnay-sous-Bois (FR)

(21) Appl. No.: 15/774,734

(22) PCT Filed: Nov. 10, 2016
(86) PCT No.: PCT/EP2016/077211

§ 371 Date: May 9, 2018

(30) Foreign Application Priority Data
Nov. 12, 2015 (FR) 1560804

(51) Int. Cl. C07C 323/41; A61K 8/46; A61Q 5/06; A61Q 5/04; A61Q 5/02; A61Q 5/12

(52) U.S. Cl. CPC C07C 323/41; A61K 8/46; A61Q 5/06; C07C 319/22; A61Q 5/02; A61Q 5/12; A61K 2800/88; A61Q 5/04

(57) ABSTRACT

The invention relates to a process for treating keratin fibres, in particular human keratin fibres such as the hair, comprising:
(i) a step of applying a compound (I)

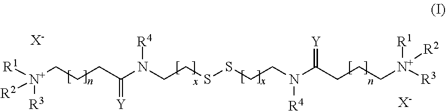

and also the acid or base salts thereof, the optical or geometrical isomers thereof,
and the solvates thereof such as hydrates,
in which compound of formula (I):
R¹, R², R³, R⁴, x, m, n, X⁻, are as defined in the description;
(ii) a step of heating the keratin fibres to a temperature of at least 100° C., preferably ranging from 100 to 250° C.;
   it is understood that steps (i) and (ii) may be performed at the same time or separately.

The process makes it possible to obtain good hair-conditioning cosmetic properties, with a long-lasting effect.

The invention also relates to the novel compounds and to a cosmetic composition comprising such a compound.

The invention relates to a cosmetic process for treating keratin fibres, in particular human keratin fibres such as the hair, using a cationic disulfide, and also to novel cationic disulfide compounds and a cosmetic composition comprising them, to the process for preparing the novel compounds and to a kit comprising them.

Hair is generally damaged and weakened by the action of external atmospheric agents such as light, sun and bad weather, and also by mechanical or chemical treatments, such as brushing, combing, dyeing, bleaching, permanent-waving, relaxing and repeated washing. Hair is thus damaged by these various factors and may in the long run become dry, coarse, brittle or dull or split or limp.

Thus, to overcome these drawbacks, it is common practice to resort to hair treatments which make use of compositions intended for conditioning the hair appropriately by giving it satisfactory cosmetic properties, especially a soft feel (the hair is no longer coarse), good disentangling properties leading to easy combing, and good manageability of the hair which is thus easy to shape.

These haircare compositions may be, for example, conditioning shampoos, hair conditioners, masks or serums.

However, the conditioning effect obtained fades out in the course of successive shampoo washes and does not show satisfactory persistence on shampooing.

There is thus a need for a process for treating keratin fibres, in particular the hair, that is capable of durably conditioning the keratin fibres, the conditioning effect being persistent after one or more shampoo washes performed on the treated keratin fibres.

The Applicant has discovered that the application to keratin fibres, in particular the hair, of cationic disulfides as defined below followed by a heating step makes it possible to obtain good hair-conditioning cosmetic properties, with a durable effect over time, especially after one or more shampoo washes.

The publication “Synthesis and characterization of novel cationic lipid and cholesterol-coated gold nanoparticles and their interactions with dipalmitoylphosphatidylcholine membranes”, S. Bhattacharya et al., Langmuir 2003, 19, pages 4439-4447 discloses the compound bis(N,N-dimethyl-N-tetradecylammonio-11-undecanoylaminoethyl) disulfide dibromide.

Patent application WO 93/16991 describes cationic disulfide surfactants comprising amide groups and ammonium groups containing a C₆-C₂₀ fatty chain, which may be used for treating the hair. It is not described to apply these compounds to the hair with a heating step.

Patent application EP 1 431 821 discloses the preparation of a conductive particle dispersion from the bis(1,1-trimethylammoniumdecanoylaminoethyl)disulphide. It describes the preparation of an adherent film for improving, at the application of energy, quality and sharpness of the image of the support of said film. It is not described to apply this compound to the hair.

Thus, one subject of the invention is a process for treating keratin fibres, in particular human keratin fibres such as the hair, comprising:
(i) a step of applying to the keratin fibres at least one disulfide compound of formula (I):

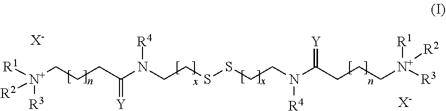

and also the acid or base salts thereof, the optical, geometrical isomers thereof, tautomers thereof, and the solvates thereof such as hydrates,
in which compound of formula (I):
R¹ denotes a hydrocarbon-based group of formula chosen from methyl, ethyl,
   i) —CH₂—(CH₂)ₘ—CH₃,
   ii) —CH₂—(CH₂)ₘ₋₁—CH(CH₃)₂, and
   iii) —CH₂—(CH₂)ₘ₋₁—C(CH₃)₃;
R², R³ denote, independently of each other, a linear hydrocarbon-based group containing from 1 to 6 carbon atoms or a branched hydrocarbon-based group containing from 3 to 6 carbon atoms, which is saturated or unsaturated, preferably saturated, optionally substituted with one or more hydroxyl groups;
R⁴ denotes a hydrogen atom or a (C₁-C₆)alkyl group, preferably a hydrogen atom;
x is an integer ranging from 1 to 20;
m is an integer ranging from 1 to 20;
n is an integer ranging from 1 to 20;
   with 10≤m+n≤30;
X⁻, which may be identical or different, represent an anionic counterion;
Y represents an oxygen or sulfur atom or a group N(R⁵) with R⁵ denoting a hydrogen atom or a (C₁-C₆)alkyl group; preferably, Y represents an oxygen atom;
(ii) a step of heating the keratin fibres to a temperature of at least 100° C., preferably ranging from 100 to 250° C.;
   it is understood that steps (i) and (ii) may be performed at the same time or separately.

The treatment process according to the invention makes it possible to obtain good keratin fibre-conditioning cosmetic properties.

In particular, the keratin fibres, especially hair, treated with the process according to the invention have a softer feel and remain disciplined. No presence of frizziness is observed. Thus, the keratin fibres are aligned, smooth and disentangle easily, which makes them easier to comb.

The process according to the invention has the advantage of giving good persistence of these good hair-conditioning cosmetic properties after one or more shampoo washes (in particular 10 and 20 shampoo washes). Thus, the treated hair is conditioned in a long-lasting manner.

A subject of the invention is also the novel compounds of formula (I′) corresponding to the compounds of formula (I) described previously, with Y preferably representing an oxygen atom;

with the exclusion of:
compounds for which x is an integer inclusively between 1 and 4; n is 1 or 2; R² and R³ represent a methyl group; and R¹ represents a C₁-C₂₀ hydrocarbon-based radical, R⁴ representing a hydrogen atom, and X is a halide ion;

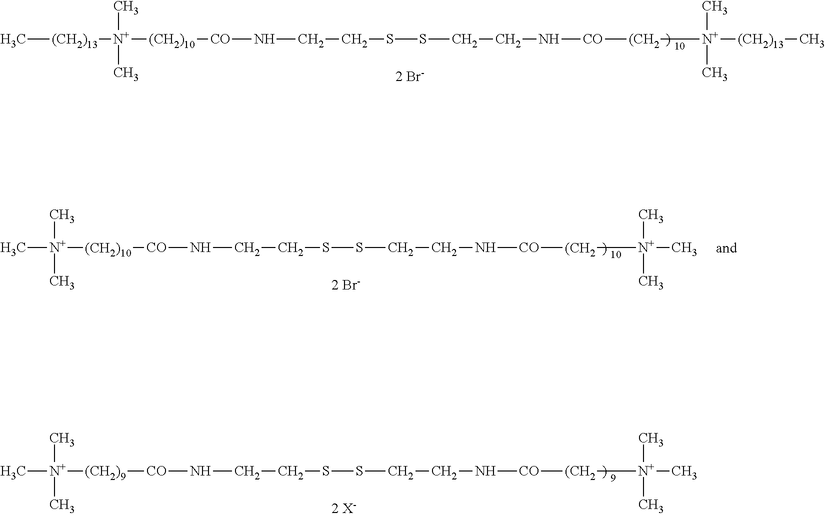

A subject of the invention is also a composition comprising, in a physiologically acceptable medium, a compound of formula (I′) as defined previously or of formula (I″), (I′″a), (I′″b) or (I′″c) as described below. Preferably, the composition comprises a physiologically acceptable aqueous medium.

A subject of the invention is also a process for preparing the compounds of general formula (I′) as defined previously.

A subject of the invention is also a kit comprising:
a cosmetic composition comprising a disulfide compound of formula (I) as defined previously, contained in a packaging assembly; and
a device for heating the keratin fibres to a temperature of at least 100° C., preferably ranging from 100 to 250° C., optionally with the application of steam, such as those described below.

The composition packaging assembly is, in a known manner, any packaging that is suitable for storing cosmetic compositions (in particular a bottle, tube, spray bottle or aerosol bottle).

Such a kit allows the process for treating keratin materials according to the invention to be performed.

For the purposes of the present invention and unless otherwise indicated:
a “hydrocarbon-based” group is a saturated or unsaturated group, i.e. a group optionally comprising one or more conjugated or unconjugated double or triple bonds, the hydrocarbon-based group is particularly saturated; linear or branched, preferably linear, said chain is optionally substituted with one or more identical or different hydroxyl groups;
an “aryl” group represents a, fused or non-fused, monocyclic or polycyclic carbon-based group comprising from 6 to 22 carbon atoms, and in which at least one ring is aromatic; preferentially, the aryl radical is a phenyl, biphenyl, naphthyl, indenyl, anthracenyl or tetrahydronaphthyl;
an “alkyl” group is a linear or branched hydrocarbon-based group, in particular a C₁-C₆, preferably C₁-C₄ hydrocarbon-based group such as methyl or ethyl;
an “alkoxy” group is an alkyl-oxy radical for which the alkyl radical is a linear or branched hydrocarbon-based radical, in particular a C₁-C₆ and preferentially C₁-C₄ hydrocarbon-based radical such as methoxy or ethoxy;
a “(poly)(hydroxy)alkyl” group is an alkyl group as defined previously which is optionally substituted with one or more hydroxyl groups;
an “organic or mineral acid salt” more particularly means the salts chosen from a salt derived from i) hydrochloric acid HCl, ii) hydrobromic acid HBr, iii) sulfuric acid H₂SO₄, iv) alkylsulfonic acids: Alk-S(O)₂OH such as methanesulfonic acid and ethanesulfonic acid; v) arylsulfonic acids: Ar—S(O)₂OH such as benzenesulfonic acid and toluenesulfonic acid; vi) (poly)(hydroxy)alkylcarboxylic acids such as citric acid; succinic acid; tartaric acid; lactic acid, x) alkoxysulfinic acids: Alk-O—S(O)OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) carboxylic acids such as acetic acid CH₃C(O)OH; xiv) triflic acid CF₃SO₃H; and xv) tetrafluoroboric acid HBF₄;
the term “anionic counterion” means an anion or an anionic group derived from an organic or mineral acid salt which counterbalances the cationic charge of the ammonium dislufide compounds; more particularly, the anionic counterion is chosen from: i) halides such as chloride or bromide; ii) nitrates; iii) sulfonates, including C₁-C₆ alkylsulfonates: Alk-S(O)₂O— such as methanesulfonate or mesylate and ethanesulfonate; iv) arylsulfonates: Ar—S(O)₂O— such as benzenesulfonate and toluenesulfonate or tosylate; v) (poly)(hydroxy)alkylcarboxylates such as citrate, succinate, tartrate, lactate; ix) alkyl sulfates: Alk-O—S(O⁻)O— such as methyl sulfate and ethyl sulfate; x) aryl sulfates: Ar—O—S(O⁻)O— such as benzene sulfate and toluene sulfate; xi) alkoxy sulfates: Alk-O—S(O)₂O— such as methoxy sulfate and ethoxy sulfate; xii) aryloxy sulfates: Ar—O—S(O)₂O—, xiii) phosphates O═P(OH)₂—O⁻O=P(O⁻)₂—OH O═P(O⁻)₃, HO—[P(O)(O—)]_{w}—P(O)(O⁻)₂ with w being an integer; xiv) acetate; xv) triflate; and xvi) borates such as tetrafluoroborate, xvii) disulfate (O═)₂S(O⁻)₂ or SO₄²⁻ and monosulfate HSO₄;
the anionic counterion, derived from the organic or mineral acid salt, ensures the electrical neutrality of the molecule; thus, it is understood that when the anion comprises several anionic charges, then the same anion may serve for the electrical neutrality of the two cationic ammonium groups in the same molecule or else may serve for the electrical neutrality of several molecules; for example, a disulfide compound of formula (I) or (I′) may contain either two “singly charged” anionic counterions 2X⁻ or a “doubly charged” anionic counterion such as (O═)₂S(O⁻)₂ or O═P(O⁻)₂—OH or tartrate —O—C(O)—CH(OH)—CH(OH)C(O)O—;
moreover, the addition salts that may be used in the context of the invention are especially chosen from addition salts with a cosmetically acceptable base such as the basifying agents as defined below, for instance alkali metal hydroxides such as sodium hydroxide, potassium hydroxide, aqueous ammonia, amines or alkanolamines;
the expression “at least one” is equivalent to “one or more”; and
the expression “inclusive” for a range of concentrations means that the limits of the range are included in the defined interval.

### The Disulfide Compound of Formula (I) or (I′):

According to a particular embodiment of the invention, the compounds of formula (I) or (I′) are such that R¹ denotes a hydrocarbon-based group:
methyl,
ethyl, and/or
i) —CH₂—(CH₂)ₘ—CH₃ with m as defined previously, preferably ethyl or i) —CH₂—(CH₂)ₘ—CH₃ with m as defined previously.

According to another particular embodiment of the invention, the compounds of formula (I) or (I′) are such that R¹ denotes a hydrocarbon-based group:

ii) —CH₂—(CH₂)ₘ₋₁—CH(CH₃)₂.

According to a particular embodiment of the invention, the compounds of formula (I) or (I′) are such that R¹ denotes a hydrocarbon-based group containing from 15 to 19 carbon atoms, i.e. in which m is an integer inclusively between 13 and 17 (13≤m≤17).

According to a particular embodiment of the invention, the compounds of formula (I) or (I′) are such that the sum of n+m is inclusively between 12 and 22, 12≤n+m≤22, and preferably are such that 14≤n+m≤18.

According to a particular embodiment of the invention, the compounds of formula (I) or (I′) are such that x is an integer inclusively between 1 and 4, and preferably x is equal to 1.

According to a particular embodiment of the invention, the compounds of formula (I) or (I′) are such that R² and R³ represent, independently of each other, a linear hydrocarbon-based group containing from 1 to 6 and preferably from 1 to 4 carbon atoms or a branched hydrocarbon-based group containing from 3 to 6 and preferably 3 or 4 carbon atoms, which is unsubstituted. Preferentially, R² and R³ denote a linear or branched (C₁-C₆)alkyl and more preferentially (C₁-C₄)alkyl group such as methyl, ethyl or isobutyl, even more preferentially methyl.

According to a particular embodiment of the invention, the compounds of formula (I) or (I′) are such that R⁴ represents a hydrogen atom.

According to a particular embodiment of the invention, the compounds of formula (I) or (I′) are such that Y represents an oxygen or sulfur atom, preferably oxygen.

According to a particular embodiment of the invention, the compounds of formula (I) or (I′) are such that X″ denotes an anionic counterion chosen from halide ions, in particular chloride, bromide, sulfate, phosphates, R″—C(O)—O— with R″ denoting an optionally hydroxylated C₁-C₅ hydrocarbon-based radical, in particular acetate, lactate, citrate, (C₁-C₄)alkyl sulfates, (C₁-C₄)alkylaryl-sulfonates, mesylate, tosylate and triflate; preferentially, X⁻ denotes a halide anionic counterion such as chloride or bromide.

According to a particular embodiment of the invention, the compounds of formula (I′) are such that n is an integer ranging from 3 to 20, better still from 3 to 15, such as 13.

According to a particular embodiment of the invention, compounds of formula (I′) of the invention are different from bis(N,N-dimethyl-N-tetradecylammonio-11-undecanoylaminoethyl) disulfide dibromide, which has the structure:

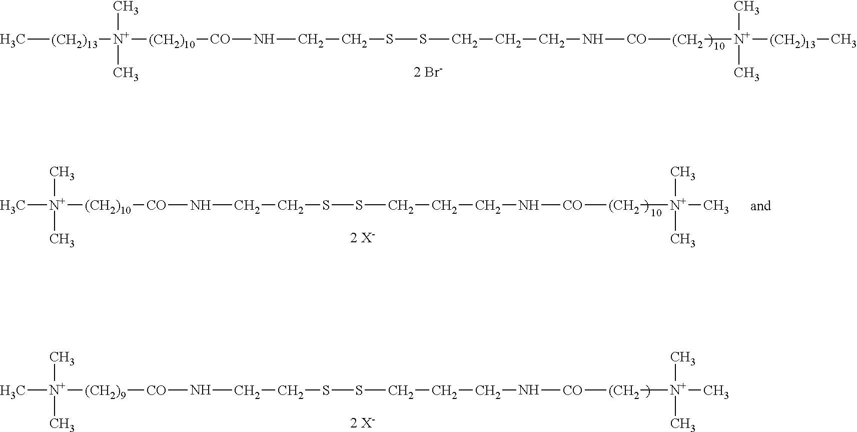

According to preferred embodiment of the invention, the compounds of formulae (I) and (I′) are chosen from:

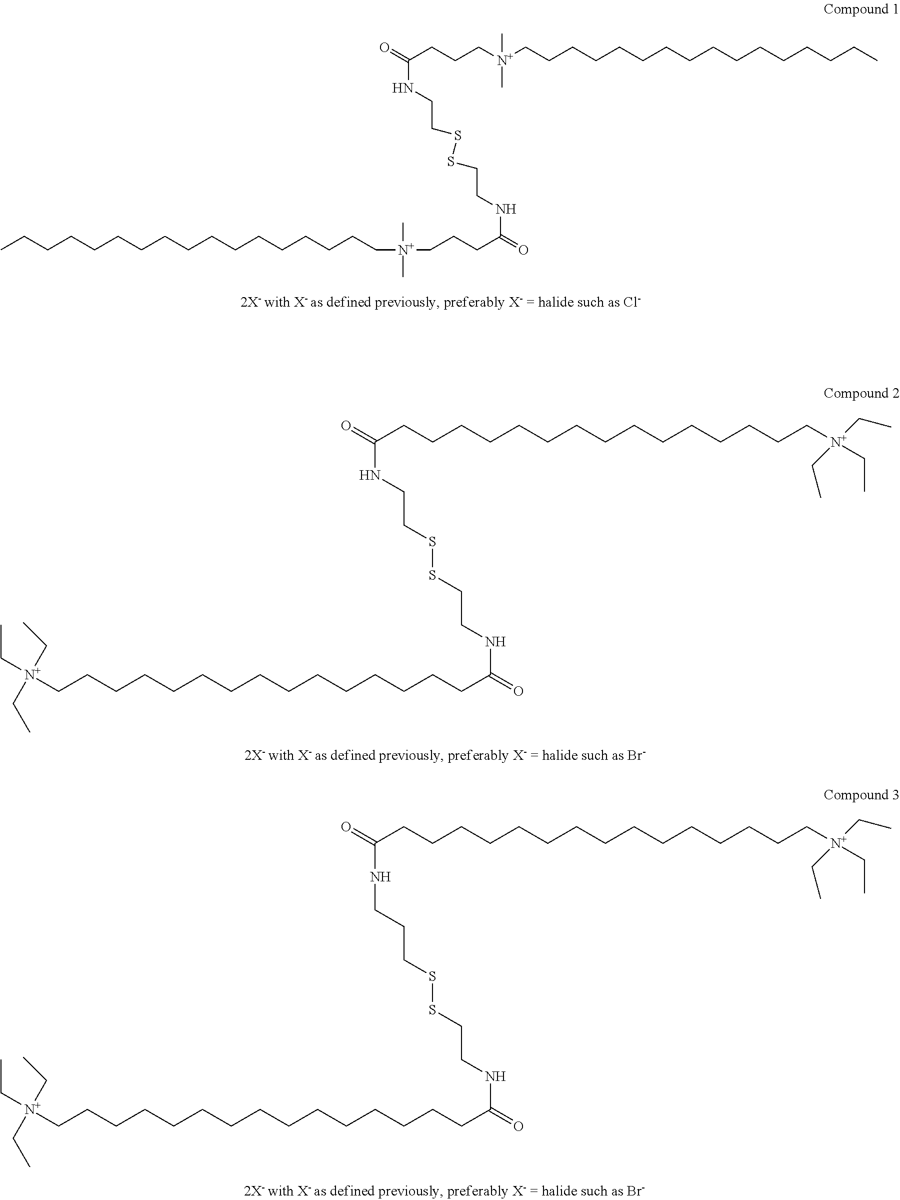 [SMILES: CCCCCCCCCCCCCCCCC[N+](C)(C)CCCC(=O)NCCSSCCNC(=O)CCC[N+](C)(C)CCCCCCCCCCCCCCCC ; CC[N+](CC)(CC)CCCCCCCCCCCCCCCC(=O)NCCSSCCNC(=O)CCCCCCCCCCCCCCC[N+](CC)(CC)CC ; CC[N+](CC)(CC)CCCCCCCCCCCCCCCC(=O)NCCCSSCCNC(=O)CCCCCCCCCCCCCCC[N+](CC)(CC)CC]

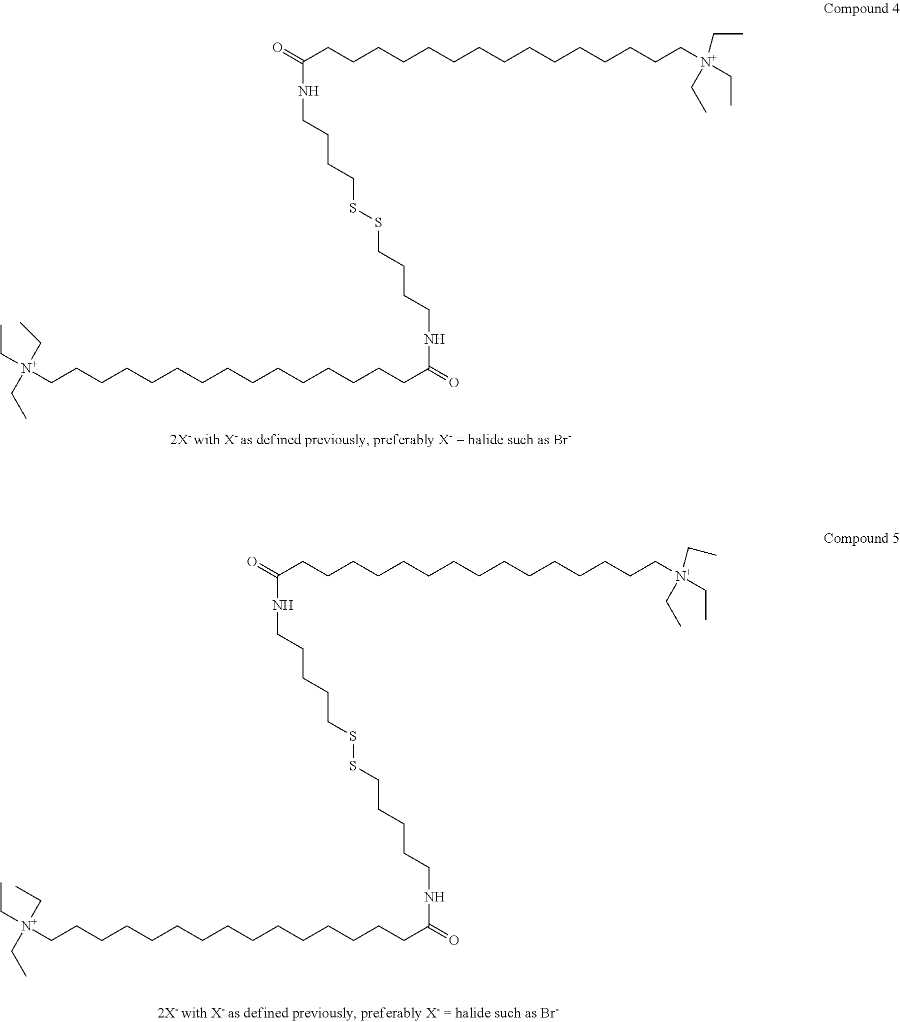 [SMILES: CC[N+](CC)(CC)CCCCCCCCCCCCCCCC(=O)NCCCCSSCCCCNC(=O)CCCCCCCCCCCCCCC[N+](CC)(CC)CC ; CC[N+](CC)(CC)CCCCCCCCCCCCCCCC(=O)NCCCCCSSCCCCCNC(=O)CCCCCCCCCCCCCCC[N+](CC)(CC)CC]

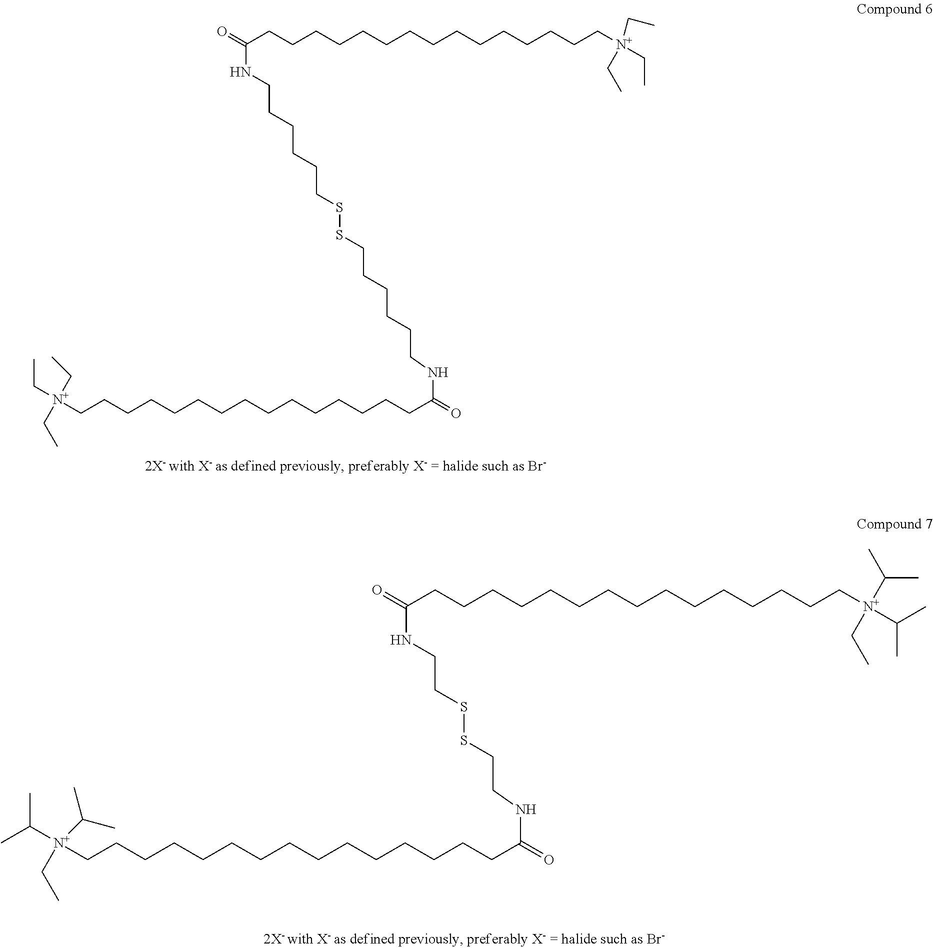

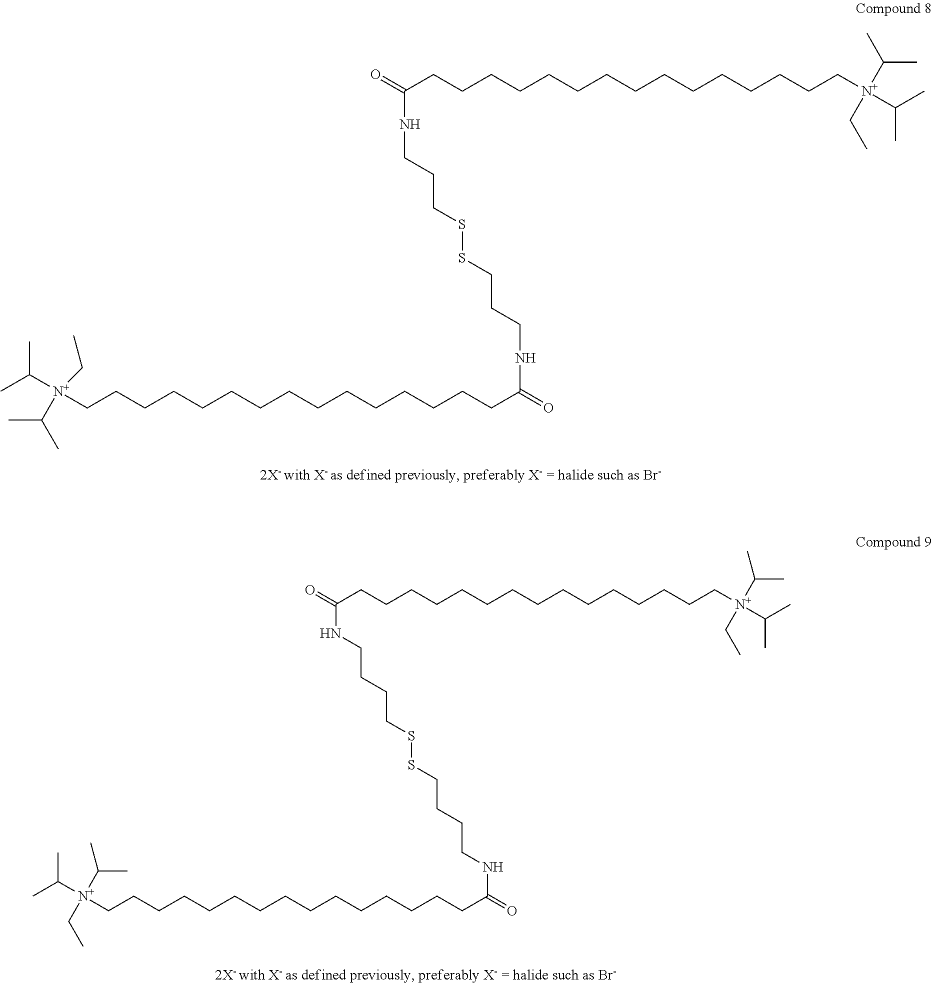

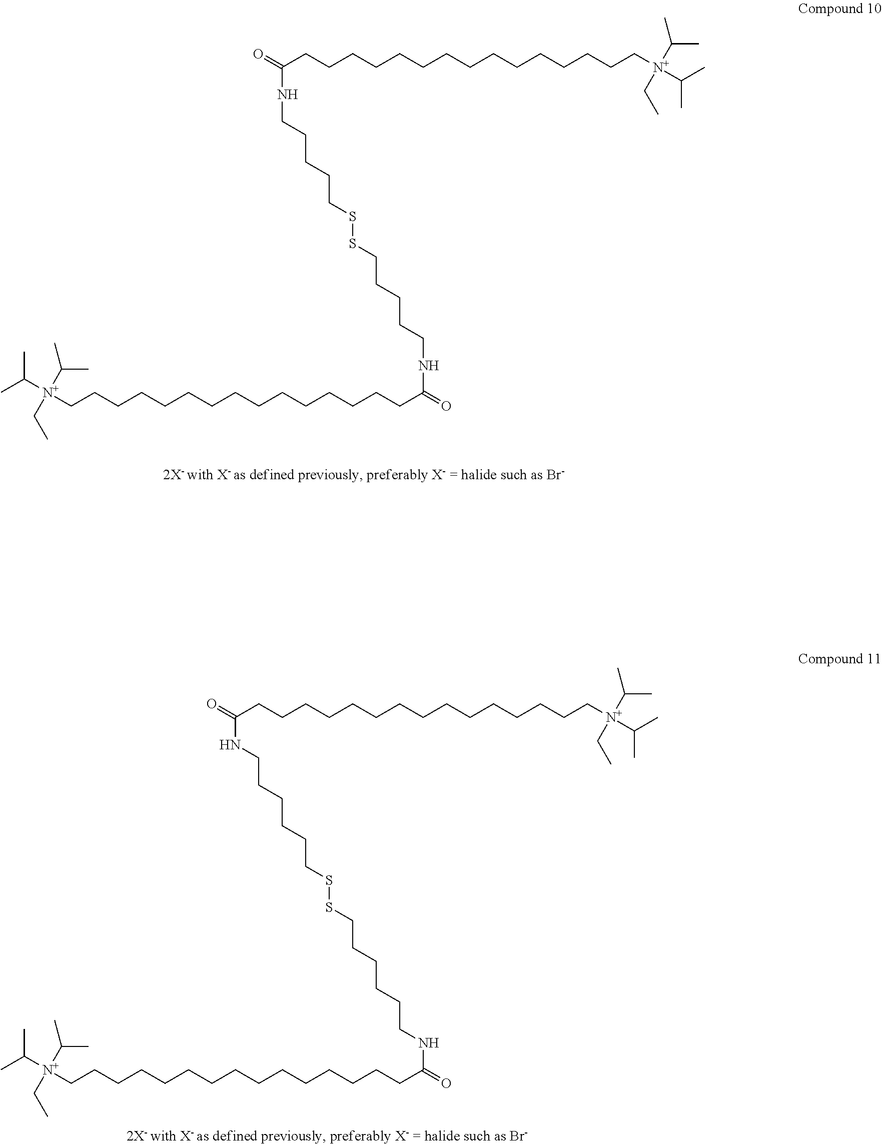

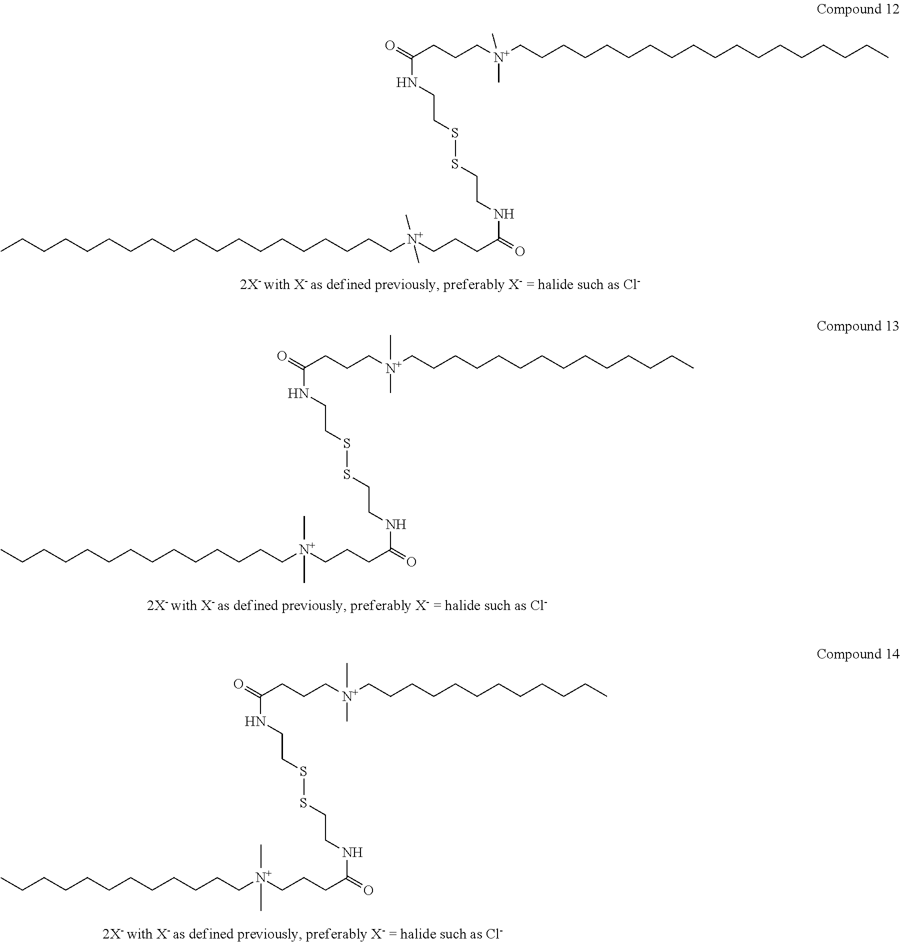

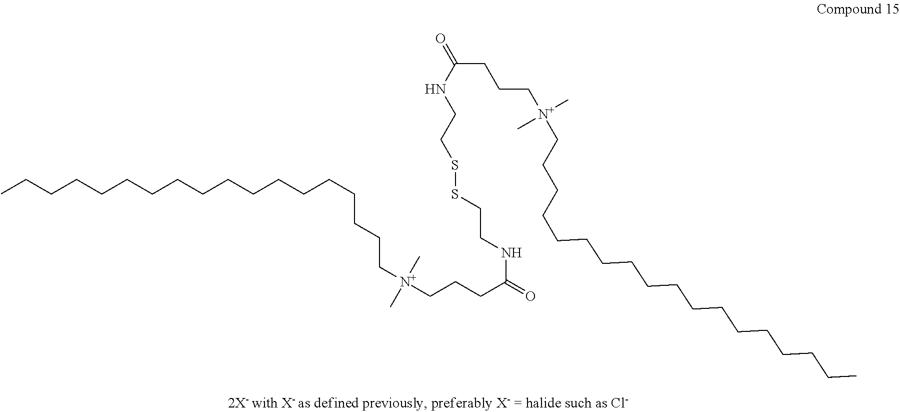 [SMILES: CCCCCCCCCCCCCCCCCC[N+](C)(C)CCCC(=O)NCCSSCCNC(=O)CCC[N+](C)(C)CCCCCCCCCCCCCCCCCC]

Advantageously, compound (I) or (I′) of the invention is Compound 1.

A subject of the invention is also the novel compounds (I″):

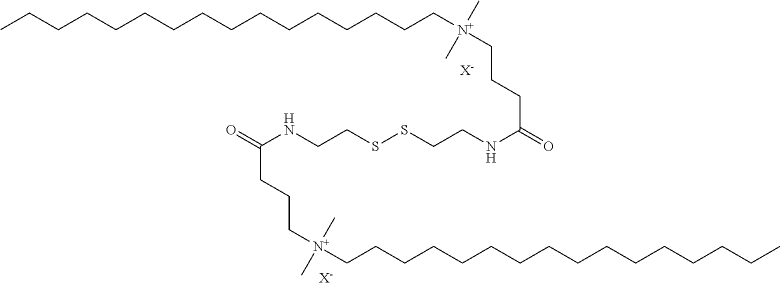

compound (I″) in which X⁻ is as defined previously, and preferably a halide such as chloride or bromide, and preferentially Cl⁻.

A subject of the invention is also the novel compounds (I′″a):

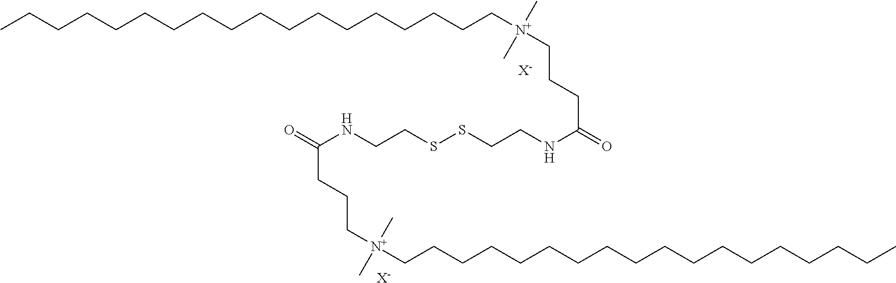

compound (I′″a) in which X is as defined previously, and preferably a halide such as chloride or bromide, and preferentially Cl⁻.

A subject of the invention is also the novel compounds (I′″b):

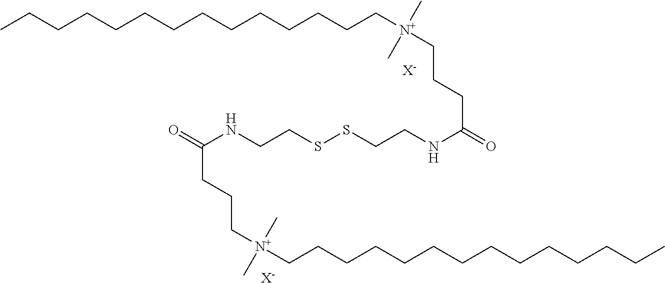

compound (I′″b) in which X⁻ is as defined previously, and preferably a halide such as chloride or bromide, and preferentially Cl⁻.

A subject of the invention is also the novel compounds (I′″c):

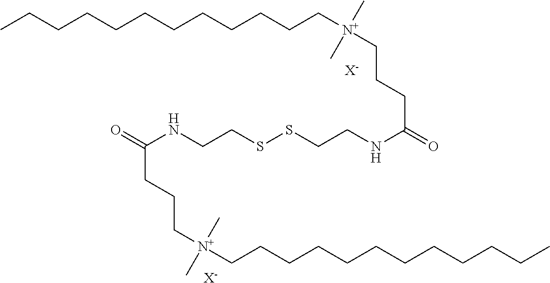

compound (I′″c) in which X is as defined previously, and preferably a halide such as chloride or bromide, and preferentially Cl⁻.

### Process for Preparing the Compounds of Formula (I) or (I′):

The compounds of formula (I) or (I′) are synthesized from the reagents that are either commercially available or synthesized via standard methods known to those skilled in the art. Mention may be made, for example, of J March's book Advanced Organic Chemistry: Reactions, Mechanisms, and Structure, 4th Ed., 1992.

The novel compounds of formula (I′), (I″), (I′″a), (I′″b) or (I′″c) for which X⁻ denotes a halide counterion may be prepared according to the following process:

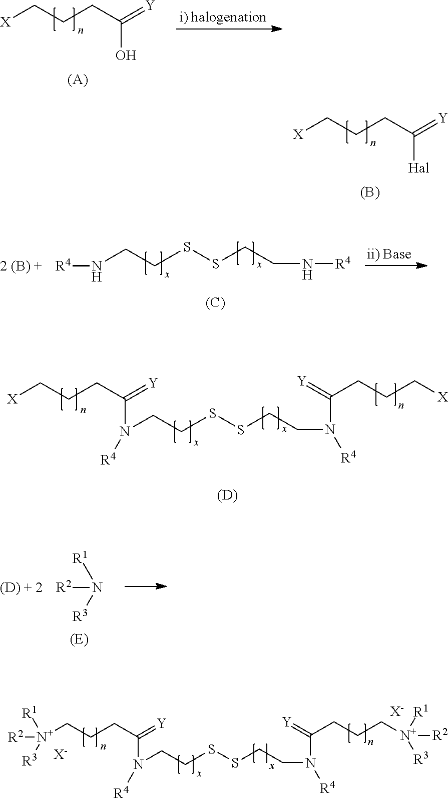

compounds (A), (B), (C), (D) and (E) with X, Y, R¹, R², R³, R⁴, n and x as defined previously for (I) or (I′) and Hal representing a halogen atom such as chlorine or bromine, preferably chlorine;

According to a first step i), a halogenation is performed, in particular chlorination of compound (A); preferably, compound (A) is a carboxylic acid, in the presence of a standard chlorination halogenating agent such as thionyl chloride SOCl₂, to obtain the corresponding halide (B).

According to a second step ii), a dicondensation (amidation, thioamidation) is performed by adding to compound (B) the diamine disulfide (C) in the presence of a base, preferably a mineral base such as sodium hydroxide, in particular in aqueous medium, and at low temperature, especially at a temperature of between 0 and 5° C., to obtain compound (D).

According to a third step iii), more than 2 molar equivalents of the trialkylamine (E) are added, preferably between 2 and 10 molar equivalents, especially at a temperature close to the boiling point of the added amine. Compound (I) or (I′) as defined previously is obtained.

Preferably, the synthetic process is performed according to synthetic scheme 1 below:

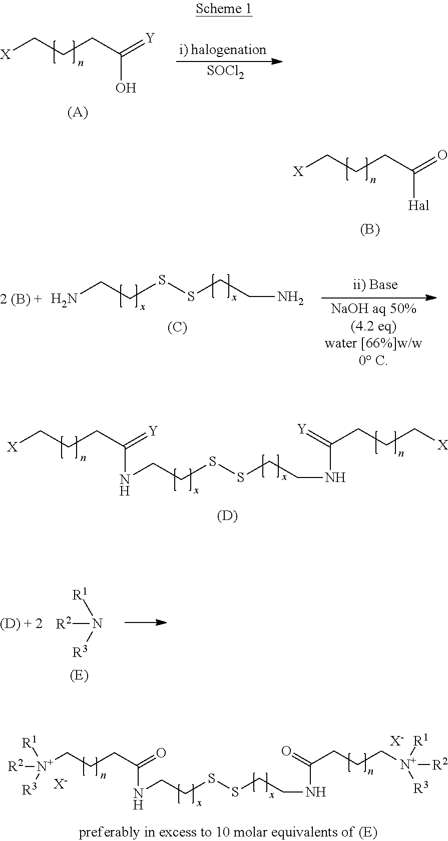

The compounds of formula (I) or (I′) may also be prepared according to synthetic scheme 2 below:

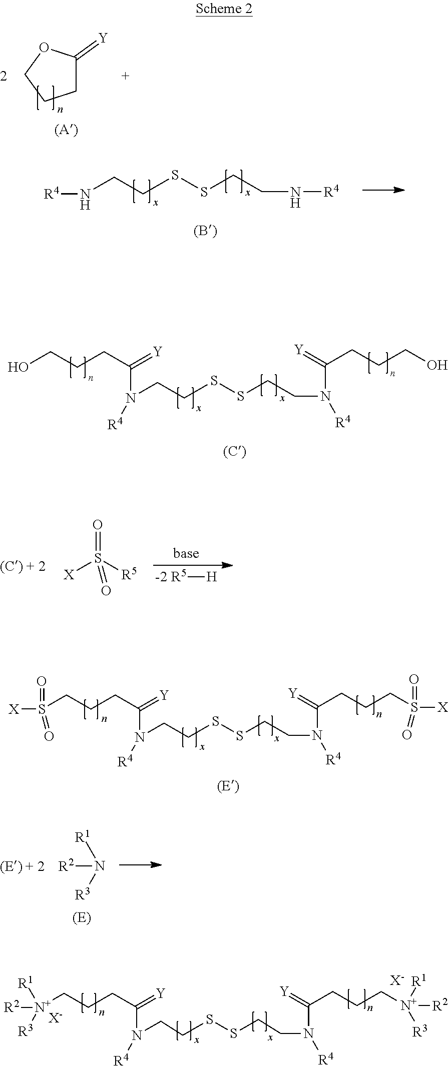

compounds (A′), (B′), (C′), (D′), (E) and (E′) with X, Y, R¹, R², R³, R⁴, n and x as defined previously for (I) or (I′) and R⁵ representing a halogen atom such as chlorine or bromine, preferably chlorine, or a (C₁-C₆)alkoxy group such as methoxy, benzyloxy, (di/tri)(halo)alkyl or (di/tri)(halo)alkoxy such as CF₃—O—.

According to a first step, condensation of the (thio)lactone (A′) with the disulfide (B′) is performed in the presence of a polar solvent such as methanol, ethanol, isopropanol or butanol at a temperature close to the boiling point of the solvent, to form compound (C′), which is isolated either by precipitation or after evaporating off the solvent. Compound (C′) is then placed in a solvent such as dichloromethane, acetonitrile, and the mixture is cooled to a temperature of between 0° C. and 5° C. A mineral base such as sodium carbonate, sodium hydrogen carbonate or an organic base such as triethylamine or diisopropylethylamine is added, followed by addition of compound D′ with an amount of base added in an equivalent amount relative to R⁵ present in (D′) if R⁵ represents a halogen atom, to obtain compound (E′). At least 2 molar equivalents and at most 10 molar equivalents of the amine (E) are then added without a solvent or in a polar solvent such as isopropyl acetate or an apolar aprotic solvent such as toluene, especially at a temperature close to the boiling point of the amine used or close to the boiling point of the solvent used, where appropriate. Compound (I) or (I′) is thus obtained. It is possible to perform an anion exchange according to the standard methods to change the anionic counterion X⁻ (for example ion-exchange resin).

### The Composition:

The cosmetic composition used according to the invention contains a physiologically acceptable medium, i.e. a medium that is compatible with human keratin materials such as the skin (of the body, face, eye contour or the scalp), the hair, the eyelashes, the eyebrows, bodily hair, the nails or the lips.

The physiologically acceptable medium of the composition used in the process according to the invention is advantageously an aqueous medium. It may be constituted, for example, of water or of a mixture of water and of at least one cosmetically acceptable organic solvent. Examples of organic solvents that may be mentioned include C2-C4 lower alcohols, such as ethanol and isopropanol; polyols, especially those containing from 2 to 6 carbon atoms, for instance glycerol, propylene glycol, butylene glycol, pentylene glycol, hexylene glycol, dipropylene glycol or diethylene glycol; polyol ethers, for instance 2-butoxyethanol, propylene glycol monomethyl ether and diethylene glycol monomethyl ether or monoethyl ether; and mixtures thereof.

Preferably, the cosmetic composition comprises from 50% to 99.5% by weight of water relative to the weight of the composition.

The composition used according to the invention may also contain one or more cosmetic additives chosen from nonionic, anionic, cationic and amphoteric surfactants, vitamins and provitamins, including panthenol, sunscreens, fillers, dyestuffs, nacreous agents, opacifiers, sequestrants, film-forming polymers, plasticizers, thickeners, oils, antioxidants, antifoams, moisturizers, emollients, penetrants, fragrances and preserving agents.

The composition used according to the invention may be in any galenical form conventionally used for application to the hair and especially in the form of aqueous solutions, aqueous-alcoholic solutions, oil-in-water (O/W), water-in-oil (W/O) or multiple (triple: W/O/W or O/W/O) emulsions, aqueous gels or aqueous-alcoholic gels. These compositions are prepared according to the usual methods. Preferably, the composition is in the form of an aqueous or aqueous-alcoholic solution or gel.

### The Process for Treating Keratin Fibres:

Process for treating keratin fibres, in particular human keratin fibres such as the hair, comprising:
(i) a step of applying to keratin fibres one or more compounds (I) or (I′) as defined previously; and
(ii) a step of heating the keratin fibres to a temperature of at least 100° C., preferably ranging from 100 to 250° C.;
   steps i) and ii) being able to be performed simultaneously or separately.

According to a particular mode of the invention, the process implements steps (i) and (ii) separately, preferably step (i) and then step (ii).

Advantageously, step (i) of applying compound (I) or (I′) consists in applying a cosmetic composition comprising the compound (I) or (I′), especially in a content ranging from 0.5% to 20% by weight, preferably ranging from 1% to 10% by weight and preferentially ranging from 2% to 5% by weight, relative to the total weight of the composition.

The process according to the invention comprises a step (ii) of heating the keratin fibres to a temperature of at least 100° C., particularly ranging from 100 to 250° C. Preferably, the step of heating the keratin fibres is performed at a temperature ranging from 170 to 250° C., preferably ranging from 180° C. to 240° C., preferentially ranging from 190° C. to 230° C., especially ranging from 200° C. to 230° C.

This heating step is advantageously performed using an iron.

The heating step is necessary for optimizing the effects of the process.

For the purposes of the present invention, the term “iron” means a device for heating keratin fibres by placing said fibres and the heating device in contact with one another.

The end of the iron which comes into contact with the keratin fibres generally has two flat surfaces. These two surfaces may be made of metal or ceramic. In particular, these two surfaces may be smooth or crimped or curved.

The heating step may be performed by means of a straightening iron, a curling iron, a crimping iron or a steam iron. Preferably, the heating step is performed using a straightening iron.

As examples of irons that may be used in the heating process according to the invention, mention may be made of any type of flat iron, and in particular, in a nonlimiting manner, those described in patents U.S. Pat. Nos. 5,957,140 and 5,046,516. The iron may be applied by successive separate strokes lasting a few seconds or by gradual movement or sliding along the locks of keratin fibres, especially of human keratin fibres such as the hair.

Preferably, the iron is applied in the process according to the invention by a continuous movement from the root to the tip of the hair, in one or more passes, in particular in two to twenty passes. The duration of each pass of the iron may last from 2 seconds to 1 minute.

According to a another particular embodiment, the process according to the invention comprises a step (ii) of applying steam. Advantageously, steam is applied to the keratin fibres, especially the hair, at a flow rate of less than 5 g/min, in particular between 1 and 4 g/min.

According to a preferred embodiment, the process according to the invention also comprises a step of applying steam. Advantageously, steam is applied to the keratin fibres, especially the hair, at a flow rate of less than 5 g/min, in particular between 1 and 4 g/min.

The application of steam may be performed using any machine known per se for generating the amount of steam of use in the process of the invention. According to a particular embodiment, this machine is portable, i.e. the tank for generating steam is in contact with the part of the device comprising the steam-dispensing orifices.

The steam application step may be performed before, during or after the heating step, and preferably before.

Preferably, the step of heating the keratin fibres is performed for a time that may range from 2 seconds to 30 minutes, and preferentially from 2 seconds to 20 minutes, better still from 2 seconds to 10 minutes, better still from 2 seconds to 5 minutes and even better still from 2 seconds to 2 minutes.

The process according to the invention may also comprise an additional step of drying the keratin fibres after step (i) of applying compound (I) or the cosmetic composition containing it and before step (ii) of heating the keratin fibres performed at a temperature of at least 100° C. The drying step may be performed using a hairdryer or a hood or by natural drying. The drying step is advantageously performed at a temperature ranging from 20 to 70° C.

According to a particular embodiment of the invention after step (i) there keratin fibers are not rinsed. Preferably after step (ii) keratin fibers are not rinsed.

After the heating step, the keratin fibres may be optionally rinsed with water or washed with a shampoo. The keratin fibers are then optionally dried with a hairdryer or a hood or in the open air.

According to one embodiment, the process according to the invention is carried out on natural keratin fibres, in particular natural hair.

According to one embodiment, the process according to the invention is performed on damaged keratin fibres, especially damaged hair. As indicated previously, the term “damaged hair” means dry or coarse or brittle or split or limp hair.

In other words, the treatment process according to the invention is preferably performed on keratin fibres, in particular sensitized hair, such as bleached, relaxed or permanent-waved fibres.

The process according to the invention may be carried out on keratin fibers, in particular hair, which is dry or wet. Preferentially, the process is carried out on dry keratin fibres, especially dry hair.

After step (i) of applying to the keratin fibres compound (I) or of a cosmetic composition containing it, and before performing step (ii) of heating the keratin fibres, compound (I) or the composition containing the same may be left on for a time ranging from 1 to 60 minutes, preferably ranging from 2 to 50 minutes and preferentially ranging from 5 to 45 minutes. The leave-on time may take place at a temperature ranging from 15° C. to 45° C., preferably at ambient temperature (25° C.).

The cosmetic composition described previously is advantageously applied to the keratin fibres in an amount ranging from 0.1 to 10 grams and preferably from 0.2 to 5 grams of composition per gram of keratin fibres.

After application of the cosmetic composition to the keratin fibres, the latter may be wrung out to remove the excess composition or washed with water or with a shampoo.

The treatment process according to the invention may be performed before, during and/or after an additional process of cosmetic treatment of the keratin fibers, such as a process for temporarily shaping (shaping with curlers, a crimping iron or a straightening iron) or a process for durably shaping (permanent-waving or relaxing) the keratin fibers.

The treatment process may be performed as a pre-treatment to a dyeing or relaxing process and/or a permanent-waving process so as to cosmetically protect the keratin fibers against these treatments. In other words, this process is performed to preserve the cosmetic properties of the keratin fibers before a cosmetic treatment process as described previously.

The treatment process according to the invention may also be performed as a post-treatment to a cosmetic treatment process, especially not leading towards artificially dyeing the keratin fibres.

In particular, the treatment process is performed as a post-treatment to a bleaching or relaxing process and/or a permanent-waving process so as to repair the said fibres.

The process according to the invention may be performed during a cosmetic treatment process so as to repair said fibres.

In particular, the treatment process according to the invention may be carried out on damaged keratin fibres.

In other words, the treatment process according to the invention is preferably performed on sensitized keratin fibres such as bleached, relaxed or permanent-waved fibres.

In particular, the treatment process may be performed before a dyeing or relaxing process and/or a permanent-waving process on keratin fibres.

As a variant, the treatment process may be used:
(a) during and/or after a process of permanent-waving or a process of relaxing keratin fibres, and
(b) after a process of bleaching keratin fibres.

According to one embodiment, the treatment process according to the invention is performed after a process of bleaching the keratin fibres.

The examples that follow are given as illustrations of the present invention.

The amounts indicated in the examples are expressed as weight percentages.

### EXAMPLE 1: PREPARATION OF COMPOUND 1

### N-(17,17-dimethyl-4,13-dioxo-8,9-dithia-5,12-diaza-17-azoniatritriacont-1-yl)-N, N-dimethylhexadecan-1-ammonium dichloride

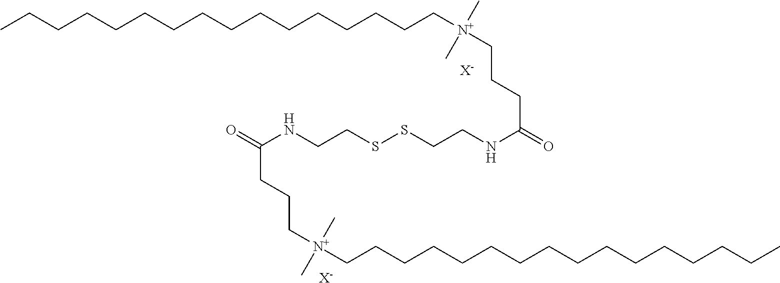

### 1st Step: Synthesis of N, N′-(disulfanediyldiethane-2,1-diyl)bis(4-chlorobutanamide)

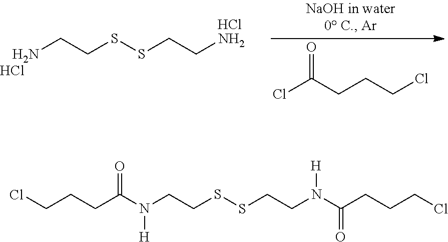 [SMILES: Cl ; Cl ; NCCSSCCN ; O=C(Cl)CCCCl ; O=C(CCCCl)NCCSSCCNC(=O)CCCCl]

107.6 g of sodium hydroxide (2.7 mol) were placed in 150 ml of water in a jacketed reactor, equipped with a condenser, an argon inlet, a temperature probe and a mechanical stirring system. The mixture was left stirring for 15 minutes at room temperature, followed by addition of 150 g of cystamine dihydrochloride (0.672 mol) and 850 ml of water. Stirring was continued for 90 minutes, setting a nominal temperature at −15° C. After 90 minutes, a temperature of −2° C. and a pH of 11.4 were reached. 1.50 ml of 4-chlorobutyryl chloride (95% tech., 1.34 mol) were added dropwise over 60-80 minutes while monitoring the addition with the temperature (<10° C.) and the pH (>9). The reaction medium was maintained at room temperature for 18 hours, while monitoring the reaction progress by TLC: 9/1 dichloromethane/methanol eluent, revelation by UV and KMnO4. The reaction medium was filtered and rinsed thoroughly with water. The precipitate was dried under vacuum at 40° C. in the presence of P₂O₅ to constant weight, to obtain 157 g of the expected compound in the form of a white powder.

NMR spectrum and mass spectrum compliant.

### 2nd Step: Synthesis of Compound 1

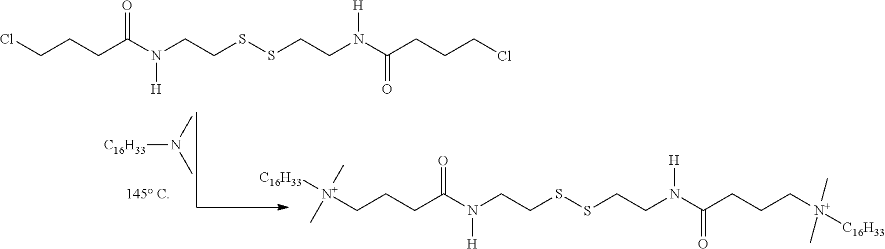

5.8 g of N,N-dimethylhexadecylamine (0.01 mol) and 3.6 g (0.02156 mol) of the compound obtained in the preceding step 1 were placed in 100 ml reactor, equipped with a condenser, an argon inlet, a temperature probe and a mechanical stirring system. The reaction medium was left stirring for 15 minutes at room temperature and was then maintained at 145° C. for 6 hours. It was then allowed to cool to room temperature and the paste obtained was taken up in 50 ml of acetone. This operation was repeated three times, removing the acetone phase each time. The paste obtained was dried under vacuum at 45° C. for 6 hours. 7 g of a brown paste were obtained.

NMR spectrum and mass spectrum compliant.

### EXAMPLE 2: PREPARATION OF COMPOUND 2

### 1st Step

10 g (0.0779 mol) of hexadecanolide lactone, 33.3 ml of aqueous sodium hydroxide at 50% by weight (33.3 mL) in 50 ml of toluene and 0.186 g of tetrabutylammonium hydrogen sulfate were placed in a three-necked round-bottomed flask. The reaction mixture was stirred for 6 hours at 95° C. After cooling, the solid formed was filtered off, rinsed several times with ether and then placed in 400 ml of distilled water and acidified with concentrated hydrochloric acid to pH 1.5. The precipitate obtained was filtered off and then rinsed several times with distilled water, and then dried under vacuum at 60° C. 11.5 g of 16-hydroxy-hexadodecanoic acid were obtained, in the form of a white solid (97% yield).

1H NMR and mass spectra compliant.

### 2nd Step

10.62 g (0.039 mol) of 16-hydroxyhexadecanoic acid were placed in a three-necked flask under nitrogen, equipped with a condenser, a mechanical stirrer and a nitrogen inlet, followed by addition of 50 ml of hydrobromic acid at 33% by weight in acetic acid and 18.6 ml of concentrated sulfuric acid.

The assembly was then connected to three traps containing sodium hydroxide, aqueous sodium hydroxide at 25% by weight and saturated potassium carbonate solution. The reaction mixture was stirred vigorously for 15 hours at 20° C. and then refluxed for 3 hours. After cooling, the reaction mixture was poured into ice-water and the precipitate formed was extracted with dichloromethane (3×100 ml). The organic phase was washed with distilled water (3×50 ml), dried over MgSO₄ and evaporated under vacuum. 11 g of 16-bromohexadecanoic acid Br—C₁₅H₃₀—C(O)OH were recovered in the form of an off-white solid.

1H NMR and mass spectra compliant.

### 3rd Step

9.4 g (0.028 mol) of 16-bromohexadecanoic acid were placed in a round-bottomed flask under nitrogen, equipped with a condenser and a magnetic stirrer, followed by addition of 6.3 ml (2.5 equivalents) of oxalyl chloride. The reaction mixture was stirred and then heated to 40° C.

After stirring for 10 minutes at 20° C., the reaction mixture was heated slightly to allow total evolution of the gas, and was then maintained at reflux for 2 hours. After cooling, the excess oxalyl chloride was evaporated off under vacuum. 9.8 g of 16-bromohexadecanoyl chloride were obtained, in the form of a brown solid.

The product is stable if it is stored protected from light, under argon and in a refrigerator.

1H NMR and mass spectra compliant.

### 4th Step

0.9 g (0.004 mol) of cystamine hydrochloride and 40 ml of anhydrous acetonitrile were placed in a round-bottomed flask under nitrogen, equipped with a magnetic stirrer and a thermometer. The reaction mixture was stirred and cooled to 0° C., followed by dropwise addition of 4 g (0.04 mol) of triethylamine, with continued stirring at 0° C. for 3 hours. Next, 3.788 g (0.0112 mol) of 16-bromohexadecanoyl chloride in acetonitrile (5 mL) were added at 0° C. and stirring was continued at 0° C. for 1 hour and then at 20° C. for 15 hours. The solvent was evaporated off under vacuum and the residue was treated with water and then extracted with chloroform (3×50 ml). The organic phase was washed with 2N hydrochloric acid (2×15 mL) and then with distilled water (2×15 mL), dried over MgSO4 and finally evaporated under vacuum. The solid obtained was purified by rinsing several times with heptane/ether (5/1 vol). 4.2 g of disulfide [Br—C₁₅H30-CONHC2H4S]2- were recovered in the form of an off-white solid (82% yield).

1H NMR and mass spectra compliant.

### 5th Step

5.0 g (0.00636 mol) of dibromo disulfide obtained in step 4 and 20 ml of anhydrous tetrahydrofuran were placed in a two-necked round-bottomed flask under nitrogen, equipped with a condenser and a thermometer. The reaction mixture was heated and stirred at 40° C. until a homogeneous solution was obtained, 150 ml of triethylamine were then added dropwise and stirring was continued at 40° C. Next, the reaction mixture was refluxed for 48 hours. The excess solvent was allowed to separate out by settling, without cooling, further triethylamine (50 mL) was added and the mixture was refluxed for 30 minutes and allowed to settle again to separate out the expected product. The residue obtained, in the form of a pasty brown product, was then treated with chloroform. This treatment led to the formation of two phases: a brown phase containing the product and the colourless organic phase containing the impurities. The brown phase was separated out using a separating funnel, rinsed twice with chloroform and dried under vacuum. The brown semi-solid product was then stirred in ether until a light-beige powder was obtained. After filtration and drying under vacuum, 6 g of the expected cationic disulfide were recovered (95% yield).

1H NMR and mass spectra compliant.

### EXAMPLE 3

### Compositions Prepared

| | Compositions | | | | |
|---|---|---|---|---|---|
| | | | | | B |
| | A1 | A2 | A3 | A4 | (placebo) |
| Compound 1* | 0.5 | 5 | 10 | 20 | 0 |
| Water | qs 100 | qs 100 | qs 100 | qs 100 | qs 100 |

*amount in g per 100 g of composition

In the application examples described below, locks of hair sensitized after bleaching were used (SA 40%). The composition to be evaluated is applied at a rate of 3 g of composition per 2.7 g of tresses. Each composition evaluated was applied to two tresses.

The composition was applied to tresses of hair and then left on for 20 minutes at room temperature (25° C.).

The tresses were towel dried and then dried using a hairdryer for 10 minutes at 60° C. (blow dried).

The locks were combed before applying:

either a straightening iron at a temperature of 210° C. by performing six continuous passes through the tresses for 15 seconds,
or a steam iron at a temperature of 210° C. (Steampod LP8500 Go/7PO device by Rowenta) performing three continuous passes through the tresses for 15 seconds,
or a hairdryer at 60° C. for 10 minutes.

To evaluate the durable (persistent) nature of the cosmetic properties of the hair tresses, they were then washed with one, ten or twenty successive shampoos respectively according to the following protocol:

The treated tresses were washed with an Ultradoux camomile shampoo by Garnier, at a rate of 0.4 g of shampoo per gram of hair, at a temperature of 38° C. Moisten the lock for 5 seconds with water. Apply the shampoo, massaging the lock from the root to the end for 15 seconds. Rinse with water for 10 seconds. Wring out. Leave the locks to dry in the open air overnight.

For 10 and 20 shampoo washes, the washes were done one after the other without drying in between.

The cosmetic properties of the locks after shampooing were then evaluated after one, ten or twenty shampoo washes respectively, especially the cosmetic feel, the manageability and the ease of combing of the locks (disentangling).

The performances were recorded as follows:

− for a bad result
+ for a poor result
++ for a moderate result
+++ for a good result

The following results were obtained:
1) After treating the keratin fibres with compositions A1 to A4, without a flat iron: Comparative

| | |
|---|---|
| Cosmetic properties | Compositions |

| evaluated | Shampoo | A1 | A2 | A3 | A4 |
|---|---|---|---|---|---|
| Softness | 0 shp | − | + | + | + |
| | 1 shp | − | − | + | + |
| Disentangling | 1 shp | − | − | + | + |
| Manageability | 1 shp | − | − | + | + |

2) After treating the keratin fibres with compositions A1 to A4, then with the iron: Invention

| With iron at 210° C. - Invention | | | | | | |
|---|---|---|---|---|---|---|
| Cosmetic properties | | | Compositions | | | |
| evaluated | Shampoos | | A1 | A2 | A3 | A4 |
| Softness | 0 | shp | ++ | ++ | − | − |
| | 1 | shp | + | +++ | ++ | − |
| | 10 | shp | − | +++ | ++ | + |
| Disentangling | 1 | shp | + | ++ | ++ | +++ |
| | 10 | shp | − | ++ | ++ | +++ |
| Manageability | 1 | shp | ++ | +++ | ++ | ++ |
| | 10 | shp | ++ | ++ | ++ | ++ |

3) After treating the keratin fibres with compositions A1 to A4, then with the steam iron: Invention

| With steam iron at 210° C. - Invention | | | | | | |
|---|---|---|---|---|---|---|
| Cosmetic properties | | | Compositions | | | |
| evaluated | Shampoos | | A1 | A2 | A3 | A4 |
| Softness | 0 | shp | ++ | ++ | − | − |
| | 1 | shp | + | +++ | ++ | − |
| | 10 | shp | − | +++ | ++ | + |
| Disentangling | 1 | shp | + | ++ | ++ | +++ |
| | 10 | shp | − | ++ | ++ | +++ |
| Manageability | 1 | shp | ++ | +++ | +++ | ++ |
| | 10 | shp | − | +++ | +++ | + |

The locks treated via the process according to the invention, with a content of compound 1 of 5 and 10% by weight and after having been shampooed one or more ten times, have better cosmetic properties in terms of soft feel, manageability, ease of combing (disentangling). These cosmetic properties thus have good persistence on shampooing. This trend was observed with the compounds of the invention even after shampooing 20 times: see the table below.

| With iron at 210° C. | | | | With steam iron at | |
|---|---|---|---|---|---|
| Cosmetic properties | | | | 210° C. | |
| evaluated | Shampoos | A2 | A3 | A2 | A3 |
| Softness | 20 shp | ++ | ++ | +++ | ++ |
| Disentangling | 20 shp | ++ | ++ | ++ | ++ |
| Manageability | 20 shp | ++ | ++ | ++ | ++ |

### EXAMPLE 4

### Composition Prepared

Compositions

| | | B |
|---|---|---|
| | A′2 | (placebo) |
| Compound 15* | 5 | 0 |
| Water | qs 100 | qs 100 |

*amount in g per 100 g of composition

The evaluation and the applying conditions of compositions A′2 and B above were made according to the same protocol used for example 3.

The performances were recorded as follows:

− for a bad result
+ for a poor result
++ for a moderate result
+++ for a good result

The following results were obtained:
1) After treating the keratin fibres with composition A′2, without iron: Comparative

| Cosmetic properties | | Composition |
|---|---|---|
| evaluated | Shampoo | A′2 |
| Softness | 0 shp | reference |
| | 1 shp | + |
| Disentangling | 1 shp | + |
| Manageability | 1 shp | + |

2) After treating the keratin fibres with composition A′2, then with the iron: Invention

| With iron at 210° C. - Invention | | |
|---|---|---|
| Cosmetic properties | | Composition |
| evaluated | Shampoos | A′2 |

| | | | |
|---|---|---|---|
| Softness | 0 | shp | reference |
| | 1 | shp | ++ |
| | 10 | shp | ++ |
| Disentangling | 1 | shp | ++ |
| | 10 | shp | ++ |
| Manageability | 1 | shp | ++ |
| | 10 | shp | ++ |

3) After treating the keratin fibres with composition A′2, then with the steam iron: Invention

| With steam iron at 210° C. - Invention | | |
|---|---|---|
| Cosmetic properties | | Composition |
| evaluated | Shampoos | A′2 |

| | | | |
|---|---|---|---|
| Softness | 0 | shp | reference |
| | 1 | shp | ++ |
| | 10 | shp | ++ |
| Disentangling | 1 | shp | ++ |
| | 10 | shp | ++ |
| Manageability | 1 | shp | ++ |
| | 10 | shp | ++ |

The locks treated via the process according to the invention, with a content of compound 15 of 5% by weight and after having been shampooed one or more ten times, have better cosmetic properties in terms of soft feel, manageability, ease of combing (disentangling). These cosmetic properties thus have good persistence on shampooing.

### EXAMPLE 5

### Friction Test:

The surface of healthy hair is perceived as smooth, as opposed to the surface of damaged hair which is perceived as coarse and rough. Different devices have been developed to measure the friction of hair fibers or swatches, at various scales. It has been reported that damaging the hair by bleaching (Scott, Robbins, J. Soc. Cosmet. Chem., 1980, 31, 179) and permanent waving (Schwartz, Knowles, J. Soc. Cosmet. Chem., 1963, 14, 455) increases its friction coefficient. As such, restoring a lower friction coefficient is a highly desirable effect of cosmetic treatments and various chemicals have been used to this effect (Evans, Wickett. 2012, Practical Modern Hair Science. Carol Stream: Allured Business Media. 562 p.). They improve the feeling of repaired hair, and facilitate grooming practices in the wet and dry state.

We have used a simple friction measurement in this study. The 3.5-cm hair bundles are fixed on the sample holder of a Zwickiline Z2.5 materials testing machine (ZWICK, Germany). A 0.5N normal force is applied to the swatches by pinching them in a clamp covered in disposable foam bands. The foam is discarded and replaced after each measurement. A constant displacement speed of 100 mm/min is imposed by the machine. The force is recorded over time and plotted as a force/displacement curve that reaches a plateau after a few seconds. The average friction force at the plateau is recorded. The impact of a conditioning treatment on the hair friction is obtained by comparing the average friction force of untreated hair swatches and the friction force of swatches treated by the compound of interest. For measurements in the wet state (wet friction force), the hair bundles are immersed in double-distilled water for 1 minute prior to performing the friction measurement as described above. Statistical significance of the results is evaluated by Student's t-test at the 0.05% threshold.

### Hair Swatches Treatment:

After the cosmetic treatments with compositions A′2 and A3 described above, the swatches are divided into 150 mg swatches, by weighting using a precision balance. The swatch is subdivided into 3.5 cm-long hair bundles by cutting using sharp disposable razor blades. A disposable cable-clamp is fastened at the middle of each hair bundle to prevent the sliding of hair fibers.

### Results:

We obtained the following results:
1) After treating the keratin fibres with composition A3, without iron: Comparative

| | |
|---|---|
| Cosmetic properties | Compositions |

| evaluated | Shampoos | B (Placebo) | A3 |
|---|---|---|---|
| wet softness and wet | 1 shp | 0.56 | 0.32 |
| untangling/ease of | 5 shp | 0.65 | 0.71 |
| combing (wet friction | | | |
| coefficient) | | | |

2) After treating the keratin fibres with composition A3, then with the iron: Invention

With iron at 210° C. - Invention

| | |
|---|---|
| Cosmetic properties | Compositions |

| evaluated | Shampoos | B (Placebo) | A3 |
|---|---|---|---|
| wet softness and wet | 1 shp | 0.62 | 0.29 |
| untangling/ease of | 5 shp | 0.69 | 0.58 |
| combing (wet friction | | | |
| coefficient) | | | |

3) After treating the keratin fibres with composition A′2, then with the iron: Invention

With iron at 210° C. - Invention

| | |
|---|---|
| Cosmetic properties | Compositions |

| evaluated | Shampoos | B (Placebo) | A′2 |
|---|---|---|---|
| wet softness and wet | 1 shp | 0.90 | 0.58 |
| untangling/ease of | 5 shp | | 0.76 |
| combing (wet friction | | | |
| coefficient) | | | |
| dry softness and dry | 1 shp | 0.68 | 0.56 |
| untangling/ease of | 5 shp | | 0.60 |
| combing (dry friction | | | |
| coefficient) | | | |

With composition A′2:
After 1 shampoo, the composition lowers the wet and dry friction forces (improves the wet and dry conditioning) as compared with the placebo swatches.
The effect still exists in the dry and wet state after 5 shampoos.

Thus, the locks treated via the process according to the invention with a content of compound 15 of 5% by weight and after having been shampooed one or five times, have better cosmetic properties in terms of wet softness, wet untangling/ease of combing, and dry softness, dry untangling/ease of combing. These cosmetic properties thus have good persistence on shampooing.

With composition A3:
After 5 shampoos, the conditioning effect is completely lost when A3 was applied without the flat iron.
After 5 shampoos, the conditioning effect is better than placebo when A3 was applied, and with the flat iron (wet friction force significantly different from control).

Thus, the locks treated via the process according to the invention with a content of compound 1 of 10% by weight and after having been shampooed one or five times, have better cosmetic properties in terms of wet softness and wet untangling/ease of combing. These cosmetic properties thus have good persistence on shampooing.

1-24. (canceled)

25. A method for treating keratin fibers, the method comprising:
(i) applying to the keratin fibers at least one disulfide compound of formula (I): 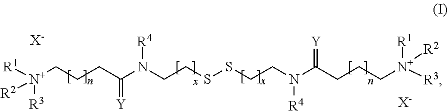
an acid or base salt thereof, an optical, geometrical isomer thereof, a tautomer thereof,
or a solvate thereof,
wherein:
R¹ is chosen from a hydrocarbon-based group chosen from methyl, ethyl,
i) —CH₂—(CH₂)ₘ—CH₃,
ii) —CH₂—(CH₂)ₘ₋₁—CH(CH₃)₂, or
iii) —CH₂—(CH₂)ₘ₋₁—C(CH₃)₃;
R² and R³, independently of each other, are chosen from a linear hydrocarbon-based group containing from 1 to 6 carbon atoms or a branched hydrocarbon-based group containing from 3 to 6 carbon atoms, which is saturated or unsaturated, and optionally substituted with at least one hydroxyl group;
R⁴ is chosen from a hydrogen atom or a (C₁-C₆)alkyl group;
x is an integer ranging from 1 to 20;
m is an integer ranging from 1 to 20;
n is an integer ranging from 1 to 20;
wherein 10≤m+n≤30;
X⁻, which may be identical or different, is an anionic counterion; and
Y is chosen from an oxygen atom, a sulfur atom, or a N(R⁵) group, wherein R⁵ is chosen from a hydrogen atom or a (C₁-C₆)alkyl group; and
(ii) heating the keratin fibers to a temperature of at least about 100° C.;
wherein steps (i) and (ii) may be performed at the same time or separately.

26. The method of claim 25, wherein R¹ is chosen from a hydrocarbon-based group:
ethyl, and/or
i) —CH₂—(CH₂)ₘ—CH₃, wherein m is an integer ranging from 1 to 20.

27. The method of claim 25, wherein R¹ is chosen from a hydrocarbon-based group ii) —CH₂—(CH₂)ₘ₋₁—CH(CH₃)₂, wherein m is an integer ranging from 1 to 20.

28. The method of claim 25, wherein R¹ is chosen from a hydrocarbon-based group containing from 15 to 19 carbon atoms.

29. The method of claim 25, wherein n is an integer ranging from 3 to 20.

30. The method of claim 25, wherein the sum of m+n ranges inclusively between 12 and 22, 12≤m+n≤22.

31. The method of claim 25, wherein x is an integer ranging inclusively between 1 and 4.

32. The method of claim 25, wherein R² and R³, which may be identical or different, are chosen from a linear hydrocarbon-based group containing from 1 to 6 carbon atoms or a branched hydrocarbon-based group containing from 3 to 6 carbon atoms, which is unsubstituted.

33. The method of claim 25, wherein R² and R³, which may be identical or different, are chosen from a linear or branched (C₁-C₆)alkyl group.

34. The method of claim 25, wherein X⁻ is an anionic counterion chosen from halide ions, chloride, bromide, sulfate, phosphates, R″—C(O)—O— with R″ denoting an optionally hydroxylated C₁-C₅ hydrocarbon-based radical, acetate, lactate, citrate, (C₁-C₄)alkyl sulfates, (C₁-C₄)alkylaryl-sulfonates, mesylate, tosylate, or triflate.

35. The method of claim 25, wherein the at least one disulfide compound of formula (I) is chosen from the following:

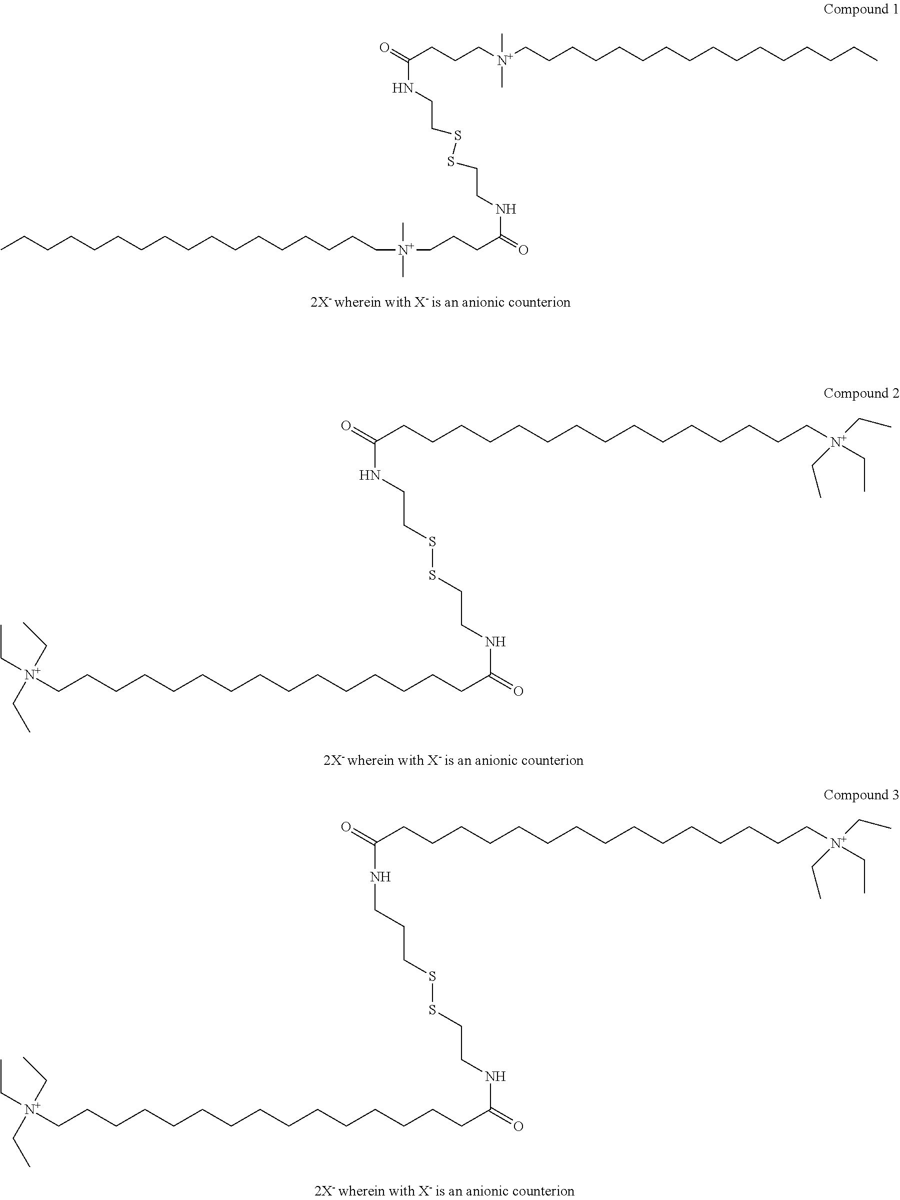 [SMILES: CCCCCCCCCCCCCCCCC[N+](C)(C)CCCC(=O)NCCSSCCNC(=O)CCC[N+](C)(C)CCCCCCCCCCCCCCCC ; CC[N+](CC)(CC)CCCCCCCCCCCCCCCC(=O)NCCSSCCNC(=O)CCCCCCCCCCCCCCC[N+](CC)(CC)CC ; CC[N+](CC)(CC)CCCCCCCCCCCCCCCC(=O)NCCCSSCCNC(=O)CCCCCCCCCCCCCCC[N+](CC)(CC)CC]

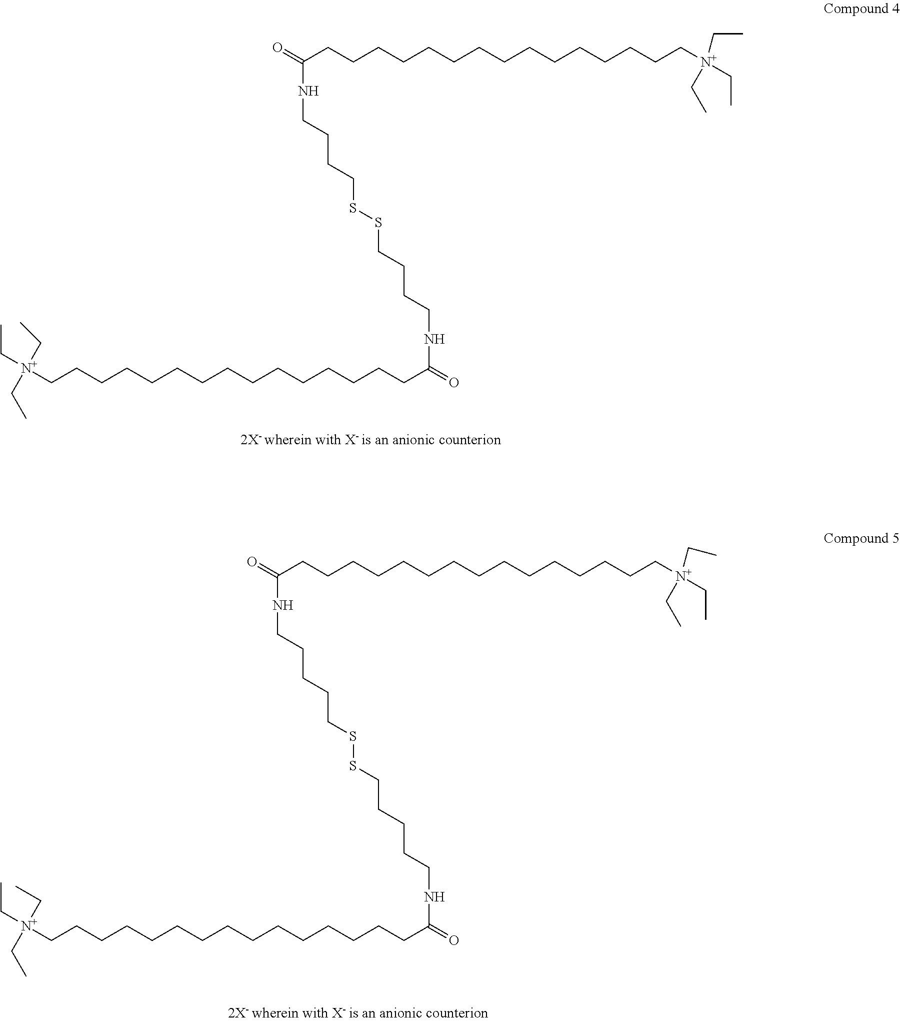 [SMILES: CC[N+](CC)(CC)CCCCCCCCCCCCCCCC(=O)NCCCCSSCCCCNC(=O)CCCCCCCCCCCCCCC[N+](CC)(CC)CC ; CC[N+](CC)(CC)CCCCCCCCCCCCCCCC(=O)NCCCCCSSCCCCCNC(=O)CCCCCCCCCCCCCCC[N+](CC)(CC)CC]

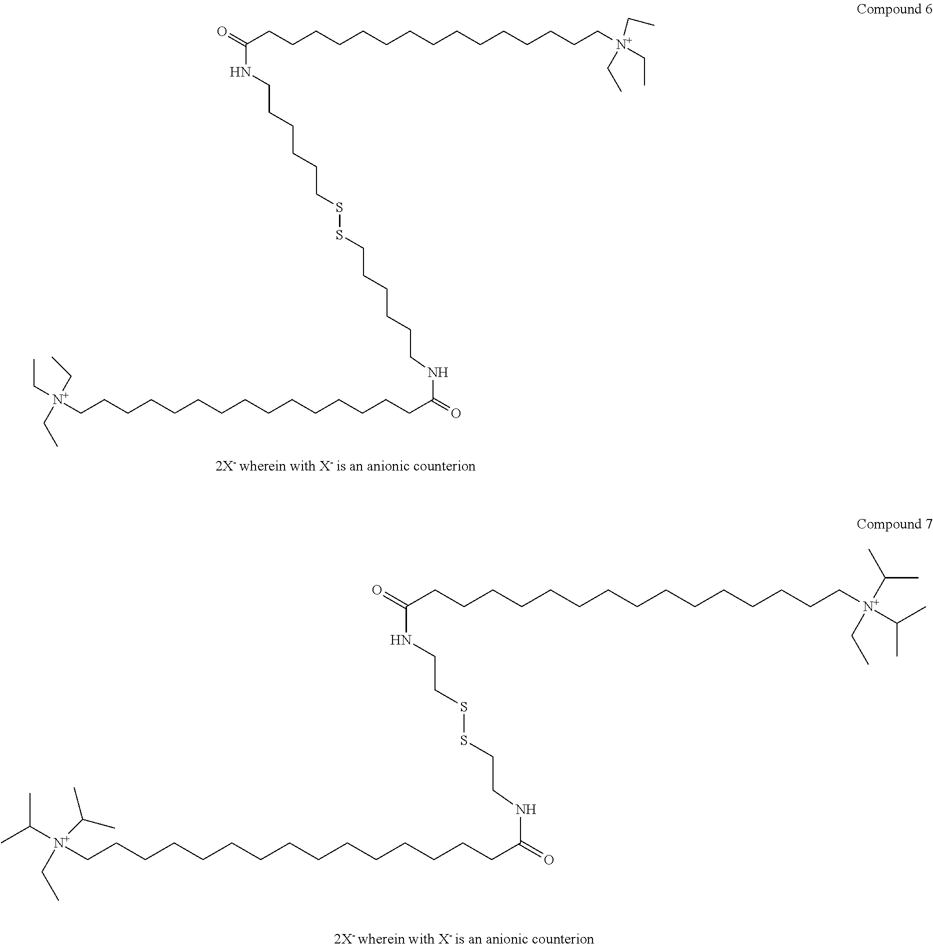

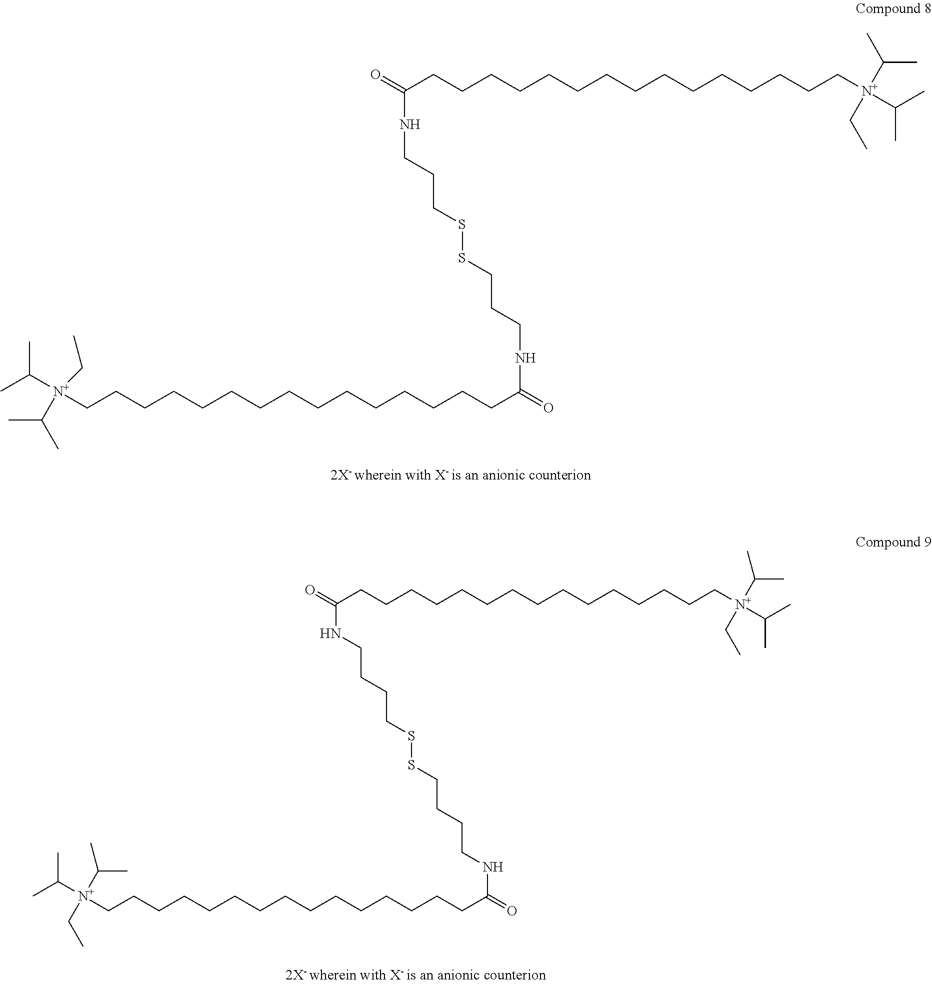

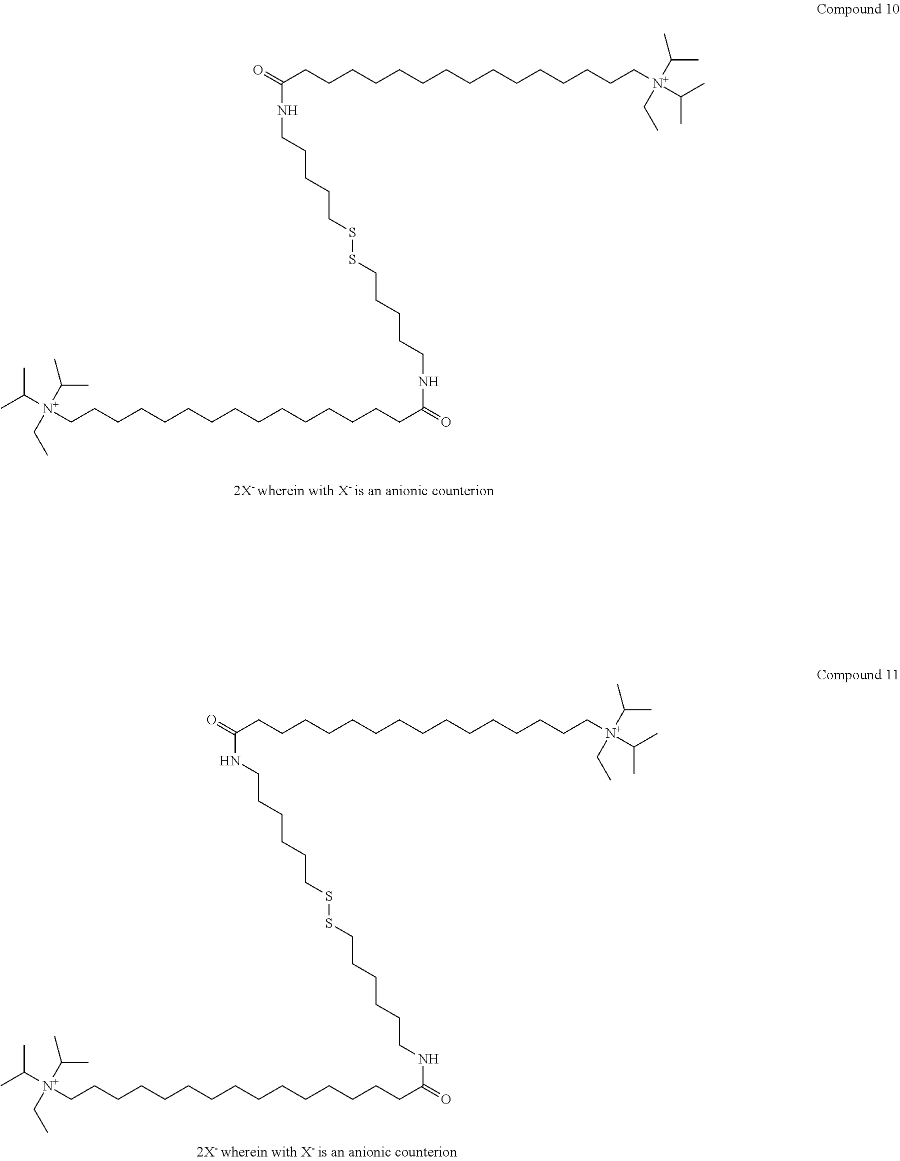

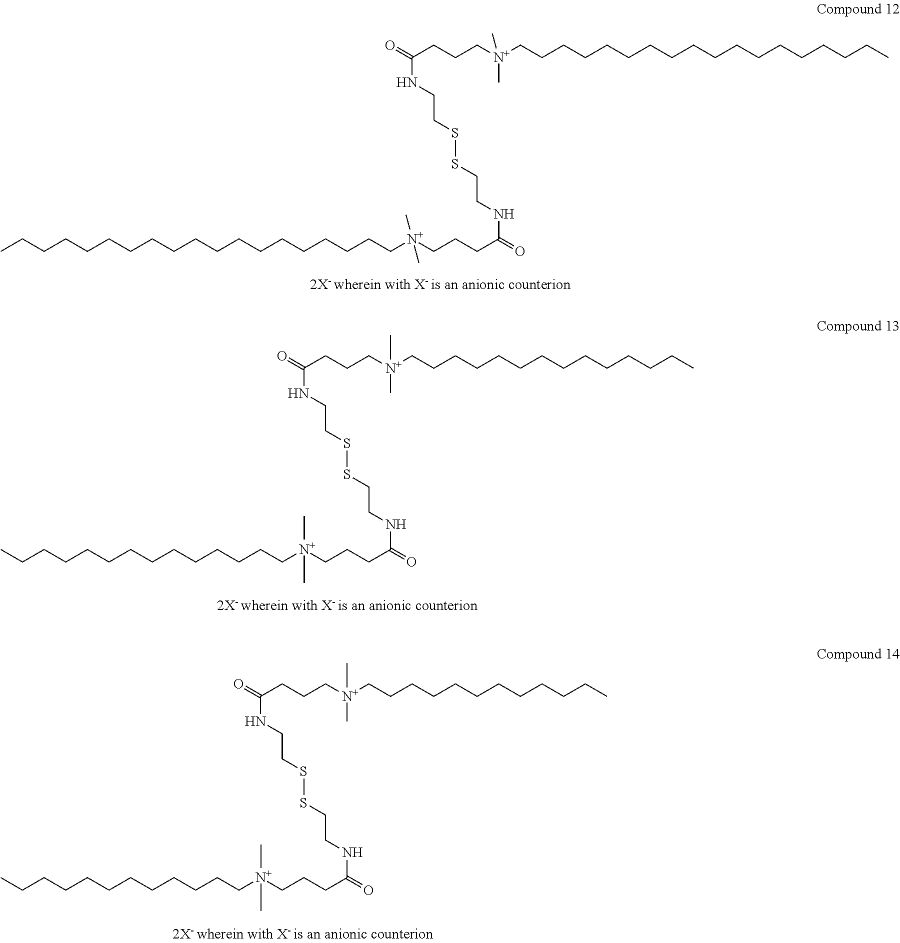

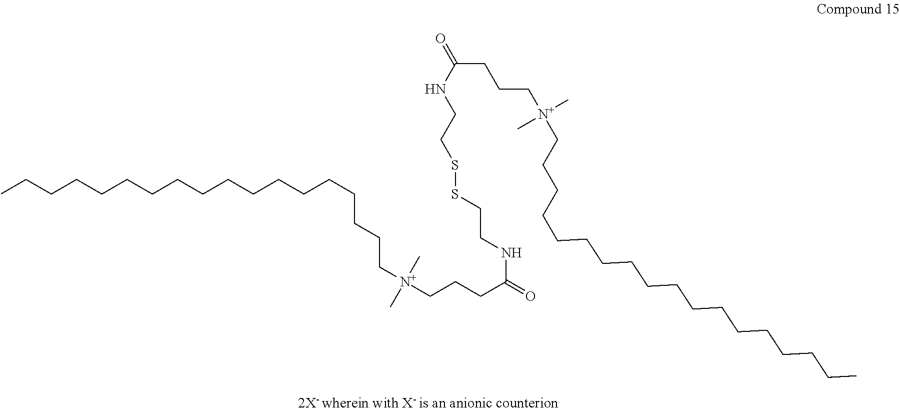 [SMILES: CCCCCCCCCCCCCCCCCC[N+](C)(C)CCCC(=O)NCCSSCCNC(=O)CCC[N+](C)(C)CCCCCCCCCCCCCCCCCC]

36. The method of claim 25, wherein the at least one disulfide compound is present in a cosmetic composition, and the at least one disulfide compound is present in an amount ranging from 0.5% to 20% by weight, relative to the total weight of the cosmetic composition.

37. The method of claim 25, wherein the heating step is performed at a temperature ranging from 100° C. to about 250° C.

38. The method of claim 25, further comprising drying the keratin fibers after the application step and before the heating step, wherein the drying step is performed at a temperature ranging from 20° C. to 70° C.

39. The method of claim 25, further comprising a leave-on time ranging from about 1 minute to about 60 minutes, after the application step and before the heating step.

40. The method of claim 25, wherein the heating step is performed with a straightening iron.

41. The method of claim 25, further comprising applying steam to the keratin fibers.

42. The method of claim 41, wherein the steam is applied at a flow rate of less than about 5 g/min.

43. The method of claim 40, wherein the straightening iron is applied to the hair in a continuous movement from the root to the end of the hairs, in at least one pass.

44. A compound of formula (I′) below: 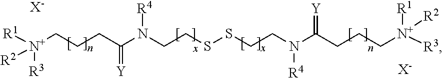
wherein:
R¹ is chosen from a hydrocarbon-based group chosen from methyl, ethyl,
i) —CH₂—(CH₂)ₘ—CH₃,
ii) —CH₂—(CH₂)ₘ₋₁—CH(CH₃)₂, or
iii) —CH₂—(CH₂)ₘ₋₁—C(CH₃)₃;
R² and R³, independently of each other, are chosen from a linear hydrocarbon-based group containing from 1 to 6 carbon atoms or a branched hydrocarbon-based group containing from 3 to 6 carbon atoms, which is saturated or unsaturated, and optionally substituted with at least one hydroxyl group;
R⁴ is chosen from a hydrogen atom or a (C₁-C₆)alkyl group;
x is an integer ranging from 1 to 20;
m is an integer ranging from 1 to 20;
n is an integer ranging from 1 to 20;
wherein 10≤m+n≤30;
X⁻, which may be identical or different, is an anionic counterion; and
Y is chosen from an oxygen atom, a sulfur atom, or a N(R⁵) group, wherein R⁵ is chosen from a hydrogen atom or a (C₁-C₆)alkyl group, with the exclusion of:
compounds wherein x is an integer inclusively between 1 and 4; n is 1 or 2; R² and R³ are a methyl group; R¹ is a C₁-C₂₀ hydrocarbon-based radical; R⁴ is a hydrogen atom; and X⁻ is a halide ion;

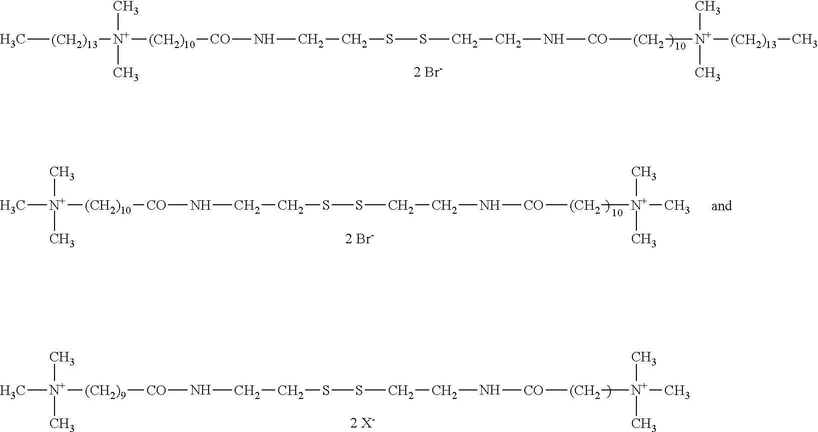

45. The compound of claim 44, chosen from: 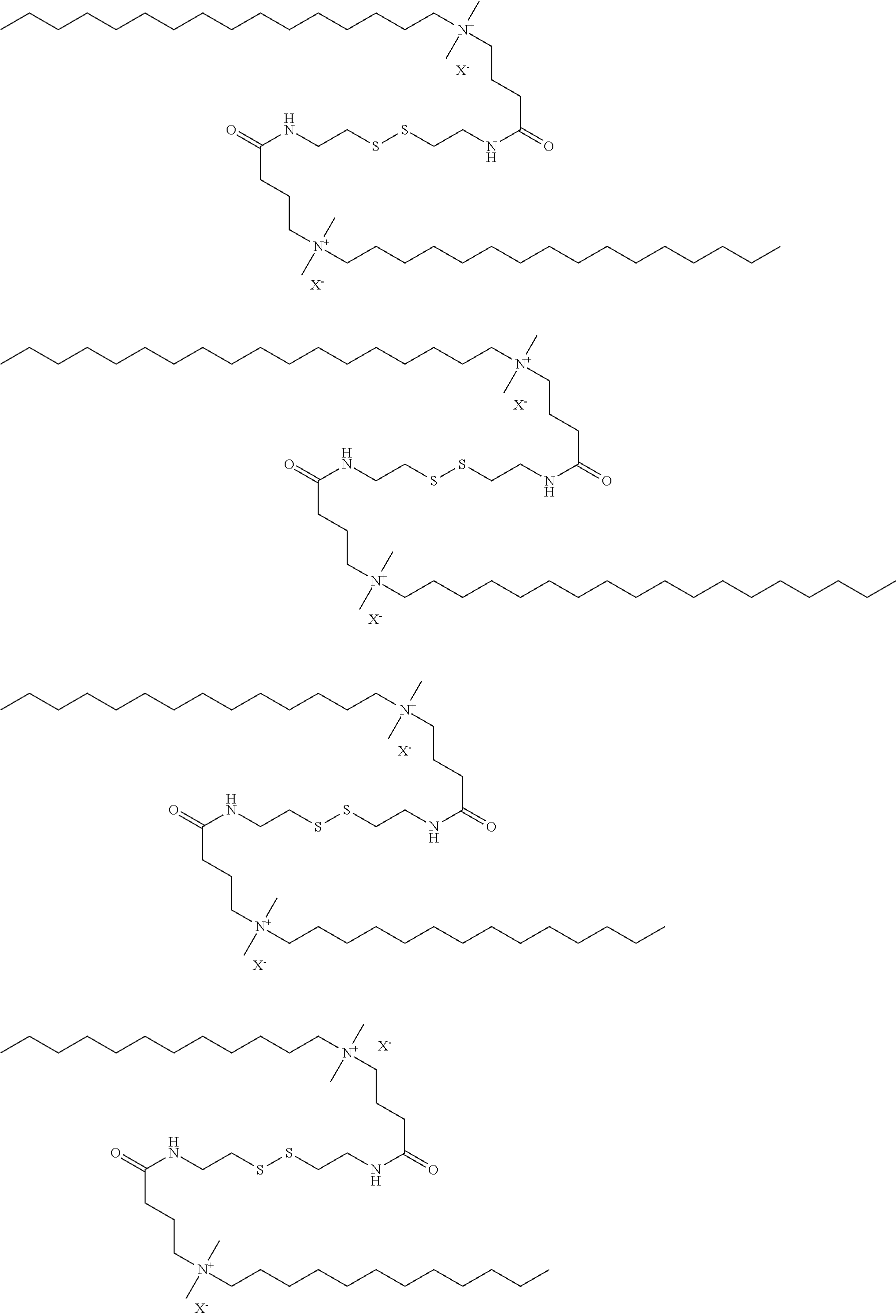
wherein X⁻, which may be identical or different, is an anionic counterion.

46. A cosmetic composition comprising, in a physiologically acceptable medium, a compound of formula (I′): 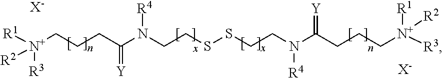
wherein:
R¹ is chosen from a hydrocarbon-based group chosen from methyl, ethyl,
i) —CH₂—(CH₂)ₘ—CH₃,
ii) —CH₂—(CH₂)ₘ₋₁—CH(CH₃)₂, or
iii) —CH₂—(CH₂)ₘ₋₁—C(CH₃)₃;
R² and R³, independently of each other, are chosen from a linear hydrocarbon-based group containing from 1 to 6 carbon atoms or a branched hydrocarbon-based group containing from 3 to 6 carbon atoms, which is saturated or unsaturated, and optionally substituted with at least one hydroxyl group;
R⁴ is chosen from a hydrogen atom or a (C₁-C₆)alkyl group;
x is an integer ranging from 1 to 20;
m is an integer ranging from 1 to 20;
n is an integer ranging from 1 to 20;
wherein 10≤m+n≤30;
X⁻, which may be identical or different, is an anionic counterion; and
Y is chosen from an oxygen atom, a sulfur atom, or a N(R⁵) group, wherein R⁵ is chosen from a hydrogen atom or a (C₁-C₆)alkyl group,
wherein the compound of formula (I′) is present in an amount ranging from about 0.5% to about 20% by weight, relative to the total weight of the composition.

47. A method for preparing compounds of formula (I′): 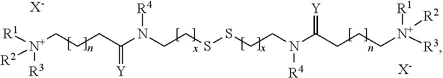
wherein:
R¹ is chosen from a hydrocarbon-based group chosen from methyl, ethyl,
i) —CH₂—(CH₂)ₘ—CH₃,
ii) —CH₂—(CH₂)ₘ₋₁—CH(CH₃)₂, or
iii) —CH₂—(CH₂)ₘ₋₁—C(CH₃)₃;
R² and R³, independently of each other, are chosen from a linear hydrocarbon-based group containing from 1 to 6 carbon atoms or a branched hydrocarbon-based group containing from 3 to 6 carbon atoms, which is saturated or unsaturated, and optionally substituted with at least one hydroxyl group;
R⁴ is chosen from a hydrogen atom or a (C₁-C₆)alkyl group;
x is an integer ranging from 1 to 20;
m is an integer ranging from 1 to 20;
n is an integer ranging from 1 to 20;
wherein 10≤m+n≤30;
X⁻, which may be identical or different, is an anionic counterion; and
Y is chosen from an oxygen atom, a sulfur atom, or a N(R⁵) group, wherein R⁵ is chosen from a hydrogen atom or a (C₁-C₆)alkyl group, according to scheme 1 or 2 below: 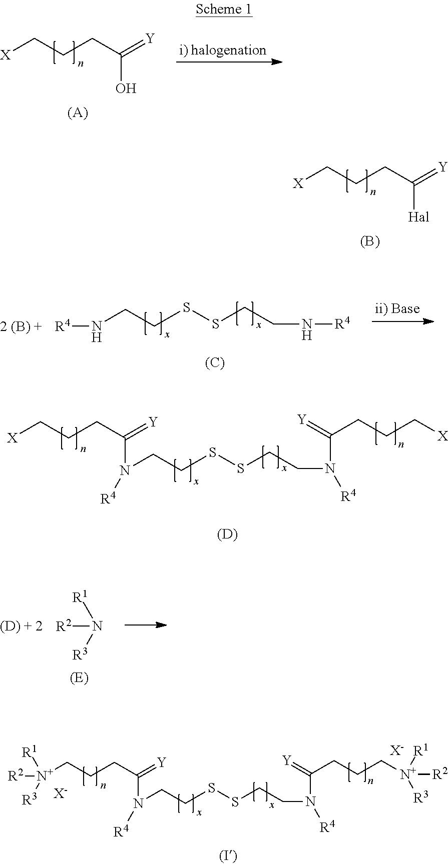
wherein compounds (A), (B), (C), (D) and (E):
X, which may be identical or different, is an anionic counterion;
Y, is chosen from an oxygen atom, a sulfur atom, or a N(R⁵) group, wherein R⁵ is chosen from a hydrogen atom or a (C₁-C₆)alkyl group;
R¹ is chosen from a hydrocarbon-based group chosen from methyl, ethyl,
i) —CH₂—(CH₂)ₘ—CH₃,
ii) —CH₂—(CH₂)ₘ₋₁—CH(CH₃)₂, or
iii) —CH₂—(CH₂)ₘ₋₁—C(CH₃)₃;
R² and R³, independently of each other, are chosen from a linear hydrocarbon-based group containing from 1 to 6 carbon atoms or a branched hydrocarbon-based group containing from 3 to 6 carbon atoms, which is saturated or unsaturated, and optionally substituted with at least one hydroxyl group;
R⁴ is chosen from a hydrogen atom or a (C₁-C₆)alkyl group;
n is an integer ranging from 1 to 20;
wherein 10≤m+n≤30;
x is an integer ranging from 1 to 20; and
Hal is a halogen atom;
wherein Scheme 1:
first, performing halogenation in the presence of a standard halogenating agent to obtain the corresponding halide (B);
second, performing dicondensation (amidation, thioamidation) by adding to compound (B) the diamine disulfide (C) in the presence of a base to obtain compound (D); and
third, adding at least 2 molar equivalents of the trialkylamine (E) to obtain the compounds of formula (I′); 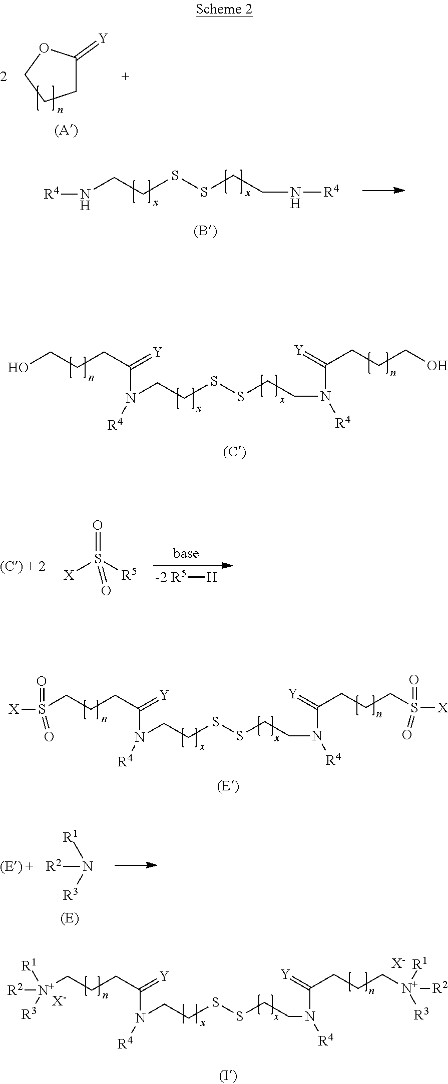
wherein compounds (A′), (B′), (C′), (D′), (E) and (E′):
X, which may be identical or different, is an anionic counterion;
Y is chosen from an oxygen atom, a sulfur atom, or a N(R⁵) group, wherein R⁵ is chosen from a hydrogen atom or a (C₁-C₆)alkyl group;
R¹ is chosen from a hydrocarbon-based group chosen from methyl, ethyl,
i) —CH₂—(CH₂)ₘ—CH₃,
ii) —CH₂—(CH₂)ₘ₋₁—CH(CH₃)₂, or
iii) —CH₂—(CH₂)ₘ₋₁—C(CH₃)₃;
R² and R³, independently of each other, are chosen from a linear hydrocarbon-based group containing from 1 to 6 carbon atoms or a branched hydrocarbon-based group containing from 3 to 6 carbon atoms, which is saturated or unsaturated, and optionally substituted with at least one hydroxyl group;
R⁴ is chosen from a hydrogen atom or a (C₁-C₆)alkyl group;
n is an integer ranging from 1 to 20;
wherein 10≤m+n≤30;
x is an integer ranging from 1 to 20; and
R⁵ is chosen from a halogen atom or a (C₁-C₆)alkoxy group;
wherein Scheme 2:
first, performing condensation of the (thio)lactone (A′) with the disulfide (B′) in the presence of a polar solvent at a temperature close to the boiling point of the solvent, to form compound (C′), which is isolated by precipitation or after evaporating off the solvent;
second, placing compound (C′) in a solvent, cooling the mixture to a temperature ranging from about 0° C. to about 5° C., adding a mineral base to the mixture, and then adding compound D′ with an amount of base in an equivalent amount relative to R⁵ present in (D′) if R⁵ is a halogen atom, to obtain compound (E′); and
third, adding the amine (E) in an amount ranging from 2 molar equivalents to 10 molar equivalents, without a solvent or in a polar solvent, to obtain compound (I′).

48. A kit comprising:
a cosmetic composition comprising a packaging assembly, wherein the packaging assembly comprises a compound of formula (I): 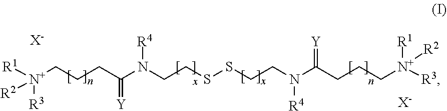
an acid or base salt thereof, an optical, geometrical isomer thereof, a tautomer thereof, or a solvate thereof,
wherein:
R¹ is chosen from a hydrocarbon-based group chosen from methyl, ethyl,
i) —CH₂—(CH₂)ₘ—CH₃,
ii) —CH₂—(CH₂)ₘ₋₁—CH(CH₃)₂, or
iii) —CH₂—(CH₂)ₘ₋₁—C(CH₃)₃;
R² and R³, independently of each other, are chosen from a linear hydrocarbon-based group containing from 1 to 6 carbon atoms or a branched hydrocarbon-based group containing from 3 to 6 carbon atoms, which is saturated or unsaturated, and optionally substituted with at least one hydroxyl group;
R⁴ is chosen from a hydrogen atom or a (C₁-C₆)alkyl group;
x is an integer ranging from 1 to 20;
m is an integer ranging from 1 to 20;
n is an integer ranging from 1 to 20;
wherein 10≤m+n≤30;
X⁻, which may be identical or different, is an anionic counterion; and
Y is chosen from an oxygen atom, a sulfur atom, or a N(R⁵) group, wherein R⁵ is chosen from a hydrogen atom or a (C₁-C₆)alkyl group, and
a device for heating the keratin fibers to a temperature of at least about 100° C.
